(19) United States
(12) Patent Application Publication
(10) Pub. No.: US 2022/0331241 A1
(43) Pub. Date: Oct. 20, 2022

(54) TREATMENT OF X-LINKED JUVENILE RETINOSCHISIS

(71) Applicant: Regeneron Pharmaceuticals, Inc., Tarrytown, NY (US)

(72) Inventors: Carmelo Romano, Tarrytown, NY (US); Yang Liu, Valhalla, NY (US); Duo Sun, Ossining, NY (US)

(21) Appl. No.: 17/719,791

(22) Filed: Apr. 13, 2022

Related U.S. Application Data
(60) Provisional application No. 63/176,009, filed on Apr. 16, 2021.

(51) Int. Cl. A61K 9/00; A61P 27/02; C07K 14/705; C12N 15/86; A61B 3/10; A61B 5/398

(52) U.S. Cl. CPC A61K 9/0048; A61P 27/02; C07K 14/705; C12N 15/86; A61B 3/102; A61B 5/398; C12N 2750/14143; C12N 2750/14171; A61K 48/00

(57) ABSTRACT

The present invention generally pertains to methods of treating X-linked juvenile retinoschisis and animal models thereof. In particular, the present invention pertains to the use of RS1 gene supplementation therapy by subretinal administration to treat X-linked juvenile retinoschisis and models thereof caused by one or more missense mutations of the RS1 gene.

### CROSS-REFERENCE TO RELATED APPLICATIONS

This application claims priority to and the benefit of U.S. Provisional Patent Application No. 63/176,009, filed Apr. 16, 2021 which is herein incorporated by reference.

### FIELD

This application relates to methods for the treatment of X-linked juvenile retinoschisis.

### BACKGROUND

X-linked retinoschisis (XLRS) is a juvenile onset progressive retinal degeneration characterized by cystic retinal lesions and macular schisis. Functional impairments in XLRS include decreased visual acuity and loss of contrast sensitivity, as well as reduced b-wave of the electroretinogram. XLRS is caused by mutations in Retinoschisin 1 (RS1), which encodes the protein retinoschisin (RS1). XLRS can be caused by deletions, insertions, splice site or missense mutations of RS1, which may affect RS1 secretion, octamerization or other functions.

Gene therapy treatments have been successfully tested in mouse models of XLRS lacking expression of RS1. However, the majority of clinical cases of XLRS are caused by missense mutations which lead to expression of dysfunctional RS1. Gene supplementation therapy for XLRS presents additional challenges compared to gene replacement therapy, and has not yet been employed.

Therefore, it will be appreciated that a need exists for methods to treat XLRS and non-human animal models of XLRS caused by missense mutations of RS1 using gene therapy.

### SUMMARY

A gene therapy treatment has been developed for the partial restoration of retinal structure and function in mouse models of XLRS. Gene supplementation by subretinal delivery of AAV RS1 was shown to restore retinal structure in Rs1 KO, C59S and R141C mice, with reduced splitting of the retinal layers relative to untreated eyes as seen by optical coherence tomography (OCT). Gene supplementation therapy also restored retinal function in each of the mouse models of XLRS as seen by electroretinography (ERG). Immunohistochemistry (IHC) staining for RS1 and cone arrestin showed that cone cells were protected in treated retinas, even outside of the area of expression of transgenic RS1. In contrast, gene supplementation by intravitreal delivery of AAV RS1 failed to restore retinal structure or function. Additionally, gene supplementation of intravitreal delivery of AAV RS1 with a promoter that was not targeted to photoreceptors failed to restore retinal structure or function. Therefore, the present disclosure demonstrates the technical advantages of selecting a particular delivery mechanism and cellular target for treatment of XLRS or models thereof.

This disclosure provides a method for treatment of X-linked juvenile retinoschisis in a human subject caused by one or more missense mutations of RS1. In some exemplary embodiments, the method comprises administering to said subject a vector including a gene encoding a functional RS1 protein.

In one aspect, said vector comprises an AAV. In a specific aspect, said AAV is selected from a group consisting of AAV2, AAV5, AAV8, AAV9, a modified version of AAV2, a modified version of AAV5, a modified version of AAV8, a modified version of AAV9, and a combination thereof. In another specific aspect, said AAV is AAV2.

In one aspect, said vector further includes a promoter, wherein said promoter drives the expression of said gene in the retina. In a specific aspect, said promoter is selected from a group consisting of a rhodopsin kinase promoter, a PR2.1 promoter, a PR1.7 promoter, or an IRBP promoter. In another specific aspect, said promoter comprises a rhodopsin kinase promoter.

In one aspect, said administration comprises subretinal injection. In another aspect, said administration comprises suprachoroidal space injection.

In one aspect, said one or more missense mutations are selected from a group consisting of L13P, C38S, C40S, C42S, C59S, C63S, E72K, S73P, C83S, W96R, R102W, R102Q, G109E, G109R, C110S, C110Y, L127P, I136T, R141H, C142S, C142R, C142W, D143V, N163Y, N179D, P192S, P192T, P193S, P203L, R213W, C219S, C219R, C219W, C219G, C223S, C223R, and C223Y. In another aspect, said one or more missense mutations comprise C59S. In yet another aspect, said one or more missense mutation comprise R141C.

In one aspect, said method further comprises assessing the restoration of retinal structure using optical coherence tomography. In another aspect, said method further comprises assessing the restoration of retinal function using electroretinography.

The disclosure provides an additional method for treatment of X-linked juvenile retinoschisis in a human subject caused by one or more missense mutations of RS1. In some exemplary embodiments, said method comprises administering to said subject an AAV2 vector including a rhodopsin kinase promoter and a gene encoding a functional RS1 protein.

In one aspect, said administration comprises subretinal injection. In another aspect, said one or more missense mutations comprise C59S. In yet another aspect, said one or more missense mutations comprise R141C.

In one aspect, said method further comprises assessing the restoration of retinal structure using optical coherence tomography. In another aspect, said method further comprises assessing the restoration of retinal function using electroretinography.

These, and other, aspects of the invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 illustrates mutations to the Rs1 gene in XLRS mouse models according to an exemplary embodiment.

FIG. 2A shows co-localization of RS1 WT (red) and mutant (green) protein, using immunocytochemistry, according to an exemplary embodiment.

FIG. 2B shows expression of RS1 variants in cell lysate of transfected HEK293 cells, using a western blot under reducing conditions, according to an exemplary embodiment.

FIG. 2C shows multimerization of RS1 variants in cell lysate of transfected HEK293 cells, using a western blot under non-reducing conditions, according to an exemplary embodiment.

FIG. 2D shows co-immunoprecipitation of RS1 variants from transfected Cho-K cells, according to an exemplary embodiment.

FIG. 3A shows RS1 expression in retinal flat mounts of Rs1 KO mice after intravitreal AAV-mediated wildtype hRS1 injection, using immunohistochemistry (IHC), according to an exemplary embodiment.

FIG. 3B shows RS1 expression in retinal cryosections of R141C mice 2 months after intravitreal injection of AAV 7m8 Rho RS1, using IHC, according to an exemplary embodiment.

FIG. 3C shows RS1 expression in retinal tissues of Rs1 KO and R141C mice 6 months after intravitreal injection, using automated western blot under reducing conditions, according to an exemplary embodiment.

FIG. 3D shows no retinal structural protection in Rs1 KO and R141C mice at 2 and 4 months after intravitreal injection, using OCT imaging, according to an exemplary embodiment.

FIG. 3E shows no retinal functional rescue in Rs1 KO mice after intravitreal injection, using ERG, according to an exemplary embodiment.

FIG. 3F shows no retinal functional rescue in R141C mice after intravitreal injection, using ERG, according to an exemplary embodiment.

FIG. 4A shows the restoration of retinal structure in the eyes of XLRS mouse models treated with gene supplementation therapy through subretinal injection, using OCT, according to an exemplary embodiment.

FIG. 4B shows the restoration of retinal function in the eyes of XLRS mouse models treated with gene supplementation therapy through subretinal injection, using dark-adapted ERG, according to an exemplary embodiment.

FIG. 4C shows the overall treatment efficacy of gene supplementation therapy in XLRS mouse models 2 months after subretinal injection, using normalized area under the curve (AUC) from dark-adapted ERG, according to an exemplary embodiment.

FIG. 5 shows variability in ERG amplitudes among mice treated with subretinal gene supplementation therapy, according to an exemplary embodiment.

FIG. 6A shows the expression of hRS1 in Rs1 KO mice treated with subretinal gene supplementation therapy, using IHC, according to an exemplary embodiment.

FIG. 6B shows the expression of hRS1 in Rs1 KO mice 6 months after treatment with subretinal gene supplementation therapy, using IHC, according to an exemplary embodiment.

FIG. 6C shows the presence of monomeric hRS1 (at ˜24 kDa) in retinal samples of XLRS mouse models treated with gene supplementation therapy, using western blot under reducing conditions, according to an exemplary embodiment.

FIG. 6D shows expression of monomeric RS1 in retinal samples of XLRS mouse models 6 months after treatment with subretinal gene supplementation therapy, using western blot in reducing conditions, according to an exemplary embodiment.

FIG. 6E shows a quantification of RS1 expression in retinal tissue of XLRS mouse models after subretinal gene supplementation therapy, using protein band densitometry, according to an exemplary embodiment.

FIG. 7A shows relative RS1 expression level in eyes after intravitreal or subretinal injection, according to an exemplary embodiment.

FIG. 7B shows structural restoration of mouse retinas after intravitreal or subretinal injection, using the Schisis Repair Score, according to an exemplary embodiment.

FIG. 7C shows functional restoration of mouse retinas after intravitreal or subretinal injection, using the AUC of scotopic ERG b-wave of treated versus untreated eye, according to an exemplary embodiment.

FIG. 7D illustrates scoring of retinal schisis using the Schisis Repair Score with OCT images, according to an exemplary embodiment.

FIG. 8A shows the expression of hRS1 (a, c) and cone arrestin (b, c) in Rs1 KO mice in eyes treated with gene supplementation therapy compared to control eyes (d), using IHC, according to an exemplary embodiment.

FIG. 8B shows the expression of hRS1 mRNA in photoreceptor, ONL, and INL in a Rs1 KO mouse eye treated with gene supplementation therapy, using RNAscope (a), and corresponding overall retinal functional rescue, using ERG (b), according to an exemplary embodiment.

FIG. 8C shows local structural protection and cone cell benefit in Rs1 KO mice in eyes treated with gene supplementation therapy compared to control eyes, using IHC, according to an exemplary embodiment.

FIG. 8D shows the expression of hRS1 in a Rs1 KO mouse eye treated with gene supplementation therapy, using IHC (a), and a lack of detection of overall retinal functional rescue in the same eye, using ERG (b), according to an exemplary embodiment.

FIG. 8E shows a correlation between photopic ERG b wave at the highest luminance and the area of arrestin signal difference compared to untreated eyes, at 10 months after subretinal injection, using a linear regression analysis, according to an exemplary embodiment.

FIG. 9A shows RS1 expression in retinal cryosections of mice subretinally injected with CAG-hRS1 or Rho-hRS1 vectors, according to an exemplary embodiment.

FIG. 9B shows RS1 expression in the eyes of mice subretinally injected with CAG-hRS1 or Rho-hRS1 vectors, using western blot under reducing conditions, according to an exemplary embodiment.

FIG. 9C shows structural rescue of retinas of mice subretinally injected with CAG-hRS1 or Rho-hRS1 vectors, according to an exemplary embodiment.

FIG. 9D shows functional rescue of retinas of mice subretinally injected with CAG-hRS1 or Rho-hRS1 vectors, according to an exemplary embodiment.

### DETAILED DESCRIPTION

X-linked retinoschisis (XLRS) is a juvenile onset progressive retinal degeneration characterized by cystic retinal lesions and by macular schisis that follows a spoked-wheel pattern (Molday et al., 2012, Prog. Retin. Eye Res., 31(3):195-212). Functional impairments in XLRS include decreased visual acuity and loss of contrast sensitivity (Forsius et al., 1973, Canad. J. Opthalmol., 8:385-393; Alexander et al., 2005, Vision Res., 45(16):2095-2107), as well as reduced b-wave of the electroretinogram (ERG) (Tanino et al., 1985, Doc. Opth., 60(2):149-161; Peachey et al., 1987, Arch. Ophthalmol., 105(4):513-516). As an X chromosome-linked trait, XLRS primarily affects males, although females with XLRS have been reported in consanguineous families (Ali et al., 2003, Amer. J Ophthalmol., 136(4):767-769; Rodriguez et al., 2005, Retina, 25(1):69-74; Saleheen et al., 2008, Canad. J. Ophthalmol. 43(5):596-599; Staffieri et al., 2015, Clin. Exp. Ophthalmol., 43(7):643-647).

XLRS is caused by mutations in Retinoschisin 1 (RS1), which encodes the 224-amino acid protein retinoschisin (RS1) (Sauer et al., 1997, Nat. Genet., 17(2):164-170). XLRS is genetically heterogeneous, with disease caused by deletions, insertions, splice site or missense mutations predicted to impact RS1 secretion, octamerization or other functions, with a corresponding manifestation in disease severity (Eksandh et al., 2000, Arch. Ophthalmol., 118(8):1098-1104; Simonelli et al., 2003, Br. J. Ophthalmol., 87(9):1130-1134; Sergeev et al., 2013, Hum. Mol. Genet., 22(23):4756-4767; Bowles et al., 2011, Invest. Ophthalmol. Vis. Sci., 52(12):9250-9256). Female carriers are typically unaffected (Molday et al., 2012; Kim et al., 2007, Doc Ophthalmol, 114:21-26). Clinical hallmarks of functional impairments in XLRS include early-onset decreased visual acuity, reduced contrast sensitivity (Alexander et al., 2005), and characteristic electronegative electroretinogram (ERG) response, in which the electropositive b-wave is disproportionately reduced compared to the electronegative a-wave (George et al., 1995, BR J Ophthalmol, 79:697-202; Tantri et al., 2004, Surv Ophtalmol, 49:214-230; Tanino et al., 1985; Peachey, et al., 1987). Structural changes include schisis or retinal splitting, retinal layer disorganization, and progressive photoreceptor degeneration seen with optical coherence tomography (OCT). The treatment options for XLRS are extremely limited. Low-vision aids can be prescribed to improve visual acuity. Retinal cavities can be managed with topical 2% dorzolamide (Apushkin and Fishman, 2006, Retina, 26:741-745; Genead et al., 2010, Arch Ophthalmol, 128:190-197; Ghajarnia and Gorin, 2007, Arch Ophthalmol, 125:571-573). However, this is not effective in all patients, the long-term utility of this treatment is unknown, and it does not treat the retinal degeneration which occurs in XLRS.

Gene supplementation therapy, which transfers a functional copy of the normal human RS1 gene, has been explored for treatment of this disease, particularly as it is a recessive and monogenic disorder. The delivery of the normal hRS1 gene to the retina of young Rs1 knockout mice resulted in long-term RS1 expression and rescued retinal structure and function (Zeng et al., 2004, Ophthalmol Vis Sci, 45:3279-3285; Kjellstrom et al., 2007, Ophthalmol Vis Sci, 48:3837-3845; Park et al., 2009, Gene Therapy, 16:916-926; Seok et al., 2005; Janssen et al., 2008, Mol Ther, 16:1010-1017; Molday et al., 2012). These findings prompted two phase 1/2 trials in humans in the USA utilizing intravitreal AAV mediated gene therapy (ClinicalTrials.gov: NCT02317887, NCT02416622). Unfortunately, little to no disease mitigation was observed (Cukras et al., 2018).

In the developing mouse retina, RS1 is reported to be expressed by multiple retinal cell types, suggesting that RS1 is required for multiple aspects of retinal development (Takada et al., 2004, Invest. Ophthalmol. Vis. Sci., 45(9):3302-3312). As development proceeds, RS1 expression becomes restricted to photoreceptors. The protein is secreted as disulfide bond-stabilized homo-octamers, which are transported to other retinal sites as double octamers (Tolun et al., 2016, Proc. Natl. Acad. Sci. U.S.A., 113(19):5287-5292). Several Rs1 mutant mouse models have been reported to date, all of which until recently have been knockouts (KO) or manifest as a null mutation (Weber et al., 2002, Proc. Natl. Acad. Sci. U.S.A., 99(9):6222-6227; Zeng et al., 2004, Invest. Ophthalmol. Vis. Sci., 45(9):3279-3285; Jablonski et al., 2005, Mol. Vision, 11:569-581; Chen et al., 2018, Front. Mol. Neurosci., 10:453). These Rs1 KO or null mouse models replicate key features of the human condition, including intraretinal schisis and a reduced ERG b-wave, and have been important for developing gene replacement therapy for XLRS to the point of a clinical trial (Bush et al., 2015, Cold Spring Harb. Persp. Med., 5(8):a017368; Cukras et al., 2018, Mol. Ther., 26(9):2282-2294).

However, only about 40% of mutations causing XLRS are expected to result in null mutations, meaning a complete loss of RS1 protein (Molday et al.). A majority of disease-causing mutations instead are missense mutations that result in the expression of a mutant RS1 protein with an amino acid substitution. A wide range of possible missense mutations are known to affect RS1 function. For example, an amino acid substitution in the signal sequence of RS1 may result in an inability of RS1 to be inserted into the membrane of the ER for secretion. Substitution of hydrophobic residues in the signal sequence with proline or with hydrophilic/charged residues may prevent the signal sequence from adopting an α-helix secondary structure required for insertion into the ER membrane. Such an amino acid substitution may be a substitution of L13, for example L13P.

Alternatively, an amino acid substitution may occur in the regions flanking the discoidin domain; specifically, the regions composed of amino acids 24-62 and the C-terminal segment, composed of amino acids 220-224. The substitution may be a substitution of cysteine at position 38, 40, 42, 59 or 223 with a non-cysteine residue, for example, Ser, Arg, Trp, Tyr or Gly. C40 is responsible for forming C40-C40 disulfide-linked dimers, and C59 and C223 form intermolecular disulfide bonds to permit assembly of RS1 dimers into an octamer. Thus, substitution of Cys at 40, 59 or 223 may have only a limited effect on protein folding and secretion, yet still result in inability of a mutant RS1 polypeptide comprising such substitution to function as a cell adhesion protein. Cysteine at position 40, 59 or 223 may be substituted with Ser, Arg, Trp, Tyr or Gly. Specific examples of substitution include C38S, C40S, C42S, C59S, C223S, C223R and C223Y.

An amino acid substitution may occur in the discoidin domain of RS1, which is composed of amino acids 63-219. A substitution in the discoidin domain may be a substitution of one of the five Cys residues in the discoidin domain: C63, C83, C110, C142, and C219. Cys63 and Cys219, and Cys110-Cys142, form two intramolecular disulfide bonds that are important for protein folding. Cysteine at one of positions 63, 83, 110, 142 or 219 may be substituted with a non-cysteine residue, for example, Ser, Arg, Trp, Tyr or Gly. Specific examples of substitution include C63S, C83S, C110S, C110Y, C142S, C142R, C142W, C219S, C219R, C219W and C219G.

A substitution in the discoidin domain may alternatively be a substitution of an amino acid residue not directly involved in formation of disulfide bonds, but important for protein folding, formation or stability of the discoidin domain, and/or intermolecular interactions among adjacent subunits. Examples of such residues include highly conserved, solvent inaccessible core residues such as E72, G109, E146, R182, and P203, as well as R141 and D143. A substitution may replace a non-cysteine residue with cysteine, which may affect thiol exchange; for example, W92C, W96C, R141C, R182C, R200C, P203C, and R209C. Additionally, a substitution may affect protein charge by eliminating or reversing the charge of amino acid residues or by replacing a non-charged residue with a charged residue without affecting thiol residues; for example, E72K, W96R, R102W, R102Q, G109E, G109R, R141H, D143V, N179D and R213W.

An amino acid substitution may affect conformational stability by insertion or removal of Pro residues; for example, S73P, L127P, P192S, P192T, P193S and P203L. A substitution may also affect the hydrophobic core of RS1 by insertion or removal of polar residues (i.e., replacing a hydrophobic residue with a polar residue or replacing a polar residue with a hydrophobic residue); for example, I136T and N163Y.

Recently, new Rs1 mutant mice were described to model XLRS caused by missense mutations (see International Patent Application Publication WO 2018/157058 A1, the entire teachings of which are herein incorporated by reference). One mouse model features a C59S mutation in RS1, which inhibits the formation of functional octamers. A second mouse model features a R141C mutation, which inhibits secretion of RS1 into the extracellular space. Both models show an early onset and severe phenotype of retinoschisis, including disruption of retinal layers and reduction of ERG b-wave, demonstrating that they are effective models of the XLRS disease state for the development of potential therapeutics.

In light of the failed clinical trials of XLRS, this disclosure sets out an evaluation of the main factors contributing to successful gene therapy for XLRS. The majority of clinical cases of XLRS are caused by missense mutations that allow translation of an altered full-length protein which is either misfolded and retained within the cell (as in the Rs1 R141C mutant model) or is secreted but is poorly- or non-functional (as in the Rs1 C59S model, which produces a dimer) (Molday et al., 2012; Wu et al., 2003; Wu et al., 2005, J Biol Chem, 280:10721-10730). Gene supplementation therapy for XLRS presents additional challenges compared to gene replacement therapy. In the absence of endogenous RS1, as is the case of Rs1 KO model, cells treated by RS1 gene therapy will express only functional RS1, forming fully functional RS1 octamers. However, in the presence of endogenous dysfunctional RS1, as is the case for missense mutations, any functional RS1 provided by gene therapy may form heteromers with dysfunctional protein subunits, potentially leading to failure of secretion, octamerization, protein complex function, or simply insufficient protein complex function to rescue the retinal damage already underway by the time of treatment.

Thus, it will be appreciated that a need exists for methods to treat XLRS and non-human animal models of XLRS caused by missense mutations of RS1 using gene therapy. Here, in three genetic mouse models, 1) a genetic knockout, lacking the Rs1 gene, 2) C59S, which is incapable of forming the native disulfide linked octamer, and 3) R141C which is intracellularly retained (Liu et al., 2019, Hum Mol Genet, 28:3072-3090), the in vitro interaction between wildtype (WT) and co-expressed variant proteins, routes of delivery, and cell specific promoters were tested. Overall, this approach allowed for the dissection of potential difficulties with achieving successful gene supplementation therapy, for example, cellular and molecular mechanisms, and biodistribution of the therapeutic transgene.

Intravitreal retinal gene therapy with AAV vectors in primates has not been demonstrated to achieve uniform delivery to any subset of retinal neurons, neither to photoreceptors deep behind the barriers of the inner limiting membrane, nerve fiber layer, and many layers of retinal neurons, nor even to ganglion cells close to the vitreal surface. For this reason, subretinal injection has been established to transduce photoreceptors (PRs) in nonhuman primate (NHP) retina (Boye et al., 2012, Hum Gene Ther, 23:1101-1115; Vandenberghe et al., 2012, Sci Transl Med, 3:88ra54; Vandenberghe et al., 2013, PloS One, 8:e53463). Most clinical ocular gene therapy programs are utilizing subretinal administration to achieve delivery to photoreceptors and RPE. This injection route has been effectively applied to target RPE in clinical trials for Leber Congenital Amaurosis (LCA2) (Maguire et al., 2008; Testa et al., 2012) and choroideremia (Patricio et al., 2018). It is unclear how much biodistribution of transgene occurs to cells outside of the bleb. In the examples described in this disclosure, the extent of disease mitigation with both intravitreal and subretinal administration is investigated.

This disclosure provides new information about the essential elements for successful gene therapy for XLRS, the relationship of therapeutic outcome to transgene delivery (for example, a description of what partial disease mitigation looks like), and clear guidance for success in the clinic.

This disclosure sets forth a novel gene therapy treatment for XLRS caused by missense mutations of RS1. Subretinal delivery of an AAV vector comprising a nucleic acid sequence encoding a functional human RS1 (hRS1) protein, operably linked to a rhodopsin kinase (Rho) promoter, was surprisingly found to restore both function and structure of treated retinas in mouse models of XLRS caused by missense mutations of RS1. This successful treatment, comparably effective to gene therapy treatment of a RS1 KO mouse model, suggests that gene supplementation therapy may be an effective therapy for treating patients suffering from XLRS.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art and where ranges are provided, endpoints are included. As used herein, the terms “include,” “includes,” and “including” are meant to be non-limiting and are understood to mean “comprise,” “comprises,” and “comprising” respectively.

As used herein, the term “gene therapy” refers to the treatment of a disease or disorder by administration of genetic material. A disease may be caused by one or more mutations in a gene that lead to a failure of the cell to express the protein encoded by the gene, or expression of a completely non-functional version of the protein. Gene therapy can be used to replace the gene of interest. This application of gene therapy is referred to herein as “gene replacement therapy.” Alternatively, a disease may be caused by one or more mutations in a gene that lead to the expression of a protein variant with decreased or altered function. Gene therapy can be used to supplement the cell with a gene encoding a functional protein, which will be co-expressed in the cell alongside the dysfunctional protein variant. This application of gene therapy is referred to herein as “gene supplementation therapy.” A gene provided by gene therapy, for example human RS1 (hRS1), may be referred to as a “transgene.”

The nucleic acid sequence encoding hRS1 may comprise the coding regions of the wildtype sequence, as set forth for example in NCBI reference sequence NG 008659. Alternatively, the coding region of the genetic material encoding a therapeutic transgene, such as RS1, may be modified to include codons that are optimized for expression in the non-human animal (see, e.g., U.S. Pat. Nos. 5,670,356 and 5,874,304, which are hereby incorporated by reference in their entirety). Codon-optimized sequences are synthetic sequences, and preferably encode the identical polypeptide (or a biologically active fragment of a full-length polypeptide which has substantially the same activity as the full-length polypeptide) encoded by the non-codon-optimized parent polynucleotide. In some embodiments, the coding region of the genetic material encoding a therapeutic transgene, in whole or in part, may include an altered sequence to optimize codon usage for a particular cell type (e.g., a rodent cell).

As used herein, the term “vector” refers to a mechanism for delivery of genetic material into cells. A vector may be, for example, a plasmid, a chromosome, an artificial chromosome, a viral vector, or naked nucleic acid (DNA or RNA). A variety of vectors have been tested for use in gene therapy, for example, viral vectors including retroviruses, lentiviruses, adenoviruses, or adeno-associated viruses (AAVs). A person of skill in the art will be able to select an appropriate vector based on, for example, the size and type genetic material to deliver, the route of administration, or the target tissue or cells. AAVs may be of particular use in practicing the method of the present invention. AAVs do not cause disease, and modified AAVs used in gene therapy lack the ability to integrate into the genome or replicate.

There exist several known variant subspecies, referred to as “serotypes”, of AAV, from AAV1 to AAV11. Different serotypes of AAV show natural tropism towards different tissue and cell types, and thus the optimal AAV serotype may be chosen for a specific application. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see, for example, GenBank Accession number U89790; GenBank Accession number J01901; GenBank Accession number AF043303; GenBank Accession number AF085716; Chiorini et al., 1997; Srivastava et al., 1983; Chiorini et al., 1999; Rutledge et al., 1998; and Wu et al., 2000. Serotypes of AAV that may be well-suited to infecting retinal cells include AAV2, AAV5, AAV8 and AAV9. Synthetic serotypes have also been designed by combining the capsid and genome of different AAV serotypes, for example AAV2/5 with the genome of AAV2 and capsid of AAV5, and may be preferred for use in the method of the present invention. Capsids of AAV vectors for gene therapy may also be selectively mutated in order to optimize infectivity or tropism towards a target tissue or cell type, for example, AAV2.7m8 or AAV8BP2. In some exemplary embodiments, the vector used in connection with the present invention comprises AAV2.

In addition to the genetic sequence of a transgene itself, the vector used in connection with the present invention may also include regulatory elements facilitating expression of a transgene, for example, a promoter sequence. An appropriate promoter sequence may be selected by a person of skill in the art in order to direct expression of the transgene in a desired tissue or cell type. Promoters useful for directing expression in retinal tissue may include, for example, rhodopsin kinase (Rho) (Khani et al., 2007, Invest. Ophthalmol. Vis. Sci., 48(9):3954-3961; Young et al., 2005, Mol. Vis., 11:1041-1051), PR2.1 (Komaromy et al., 2008, Gene Ther., 15(14):1049-1055), PR1.7 (Ye et al., 2016, Hum. Gene Ther., 27(1):72-82), or interphotoreceptor retinoid-binding protein (IRBP) (Boyd et al., 2015, Gene Ther., 23(2):223-230). In some exemplary embodiments, the vector used in connection with the present invention includes a rhodopsin kinase promoter. In some exemplary embodiments, the vector used in connection with the present invention comprises AAV2 7m8-Rho-hRS1.

In some exemplary embodiments, a formulation comprising a vector used in connection with the present invention may be formulated at a titer of about 3×10¹³ vg/mL (vector genomes/mL). In some exemplary embodiments, a volume of a formulation comprising a vector used in connection with the present invention that is administered to a subject may be about 1 μL. In some exemplary embodiments, an amount of a vector used in connection with the present invention that is administered to a subject may be about 3×10¹⁰ vg/eye. A person of skill in the art may select a convenient and effective titer, volume and amount of the aforementioned species for administration to a subject.

Administration of a retinal gene therapy may be accomplished by several methods, for example intravitreal injection, suprachoroidal space injection, or subretinal injection. A person of skill in the art may select the route of administration best suited for their purpose, and will be capable of carrying out the selected injection.

Subretinal injections are injections into the subretinal space, underneath the neurosensory retina. During a subretinal injection, the injected material is directed into, and creates a space between, the photoreceptor cell and retinal pigment epithelial layers. When the injection is carried out through a small retinotomy, a retinal detachment may be created. The detached, raised layer of the retina that is generated by the injected material is referred to as a “bleb”.

The hole created by the subretinal injection must be sufficiently small that the injected solution does not significantly reflux back into the vitreous cavity after administration. Such reflux would be particularly problematic when a medicament is injected, because the effects of the medicament would be directed away from the target zone. Preferably, the injection creates a self-sealing entry point in the neurosensory retina: once the injection needle is removed, the hole created by the needle should reseal such that very little or substantially no injected material is released through the hole. To facilitate this process, specialist subretinal injection needles are commercially available (for example, DORC 41G Teflon subretinal injection needle, Dutch Ophthalmic Research Center International BV, Zuidland, The Netherlands). These are needles designed to carry out subretinal injections.

Unless damage to the retina occurs during the injection, and as long as a sufficiently small needle is used, substantially all injected material remains localized between the detached neurosensory retina and the retinal pigment epithelium at the site of the localized retinal detachment, and does not reflux into the vitreous cavity. The typical persistence of the bleb over a short time frame indicates that there is usually little escape of the injected material into the vitreous. The bleb may dissipate over a longer time frame as the injected material is absorbed.

Alternatively, a two-step subretinal injection method may be used in which a localized retinal detachment is created by the subretinal injection of a first solution. The first solution does not comprise the vector. A second subretinal injection is then used to deliver the medicament comprising the vector into the subretinal fluid of the bleb created by the first subretinal injection. Because the injection delivering the medicament is not being used to detach the retina, a specific volume of solution may be injected in this second step.

The volume of solution injected to at least partially detach the retina may be, for example, about 10-1000 μL, for example about 50-1000, 100-1000, 250-1000, 500-1000, 10-500, 50-500, 100-500, 250-500 μL. The volume may be, for example, about 10, 50, 100, 200, 300, 400, 500, 600, 700, 800, 900 or 1000 μL.

The volume of the therapeutic composition injected after first injection may be, for example, about 10-500 μL, for example about 50-500, 100-500, 200-500, 300-500, 400-500, 50-250, 100-250, 200-250 or 50-150 μL. The volume may be, for example, about 10, 50, 100, 150, 200, 250, 300, 350, 400, 450 or 500 μL. Preferably, the volume of the therapeutic composition injected is 100 μL. Larger volumes may increase the risk of stretching the retina, while smaller volumes may be difficult to see.

The solution that does not comprise the therapeutic may be similarly formulated to the solution that does comprise the therapeutic. A preferred solution that does not comprise the therapeutic is balanced saline solution (BSS) or a similar buffer solution matched to the pH and osmolality of the subretinal space.

A therapeutic formulation comprising a gene therapy vector may be formulated into a pharmaceutical composition. Such a composition may additionally comprise, for example, a pharmaceutically acceptable carrier, diluent, excipient, buffer, stabilizer or other materials well known in the art. Such materials should be non-toxic and should not interfere with the efficacy of the active ingredient. The precise nature of the carrier or other material may be determined by the skilled person according to the route of administration.

The method of the present invention may reduce or prevent the appearance of XLRS phenotypes, as described above, in a subject. For example, it may result in the protection of rod and/or cone photoreceptor cells. Numbers of rods and cones can be evaluated by the skilled person in the clinic using techniques such as adaptive optics, autofluorescence and optical coherence tomography. The method of the present invention may also result in the maintenance or improvement in visual function. Visual function tests that might be carried out by a skilled person include, for example, best corrected visual acuity, visual field testing, micro perimetry, color vision, dark adaptometry, cone flicker fusion test, visual evoked potential test, multifocal visual evoked potential test, and electroretinography.

As used herein, the term “electroretinography” (ERG) refers to a technique used for clinical assessment of retinal function. An electroretinogram (also referred to as ERG) measures the electrical activity of the retina in response to a light stimulus. The ERG arises from currents generated directly by retinal neurons in combination with contributions from retinal glia. ERG allows for non-invasive measurement of retinal function that can be recorded under physiological conditions. The ERG can be elicited by diffuse flashes or patterned stimuli. Two types of measurements that can be made by ERG include photopic measurements, which measure cone-pathway function and are recorded from a light-adapted eye, and scotopic measurements, which measure rod-pathway function and are recorded from a dark-adapted eye.

Under dark-adapted conditions, a weak, diffuse, full-field flash of light will elicit a slow cornea-positive potential called the “b-wave”. A stronger flash of light will elicit a rapid cornea-negative potential called the “a-wave”, and a subsequent b-wave. In some embodiments, the amplitude of the b-wave in response to stimulus of increasing luminance is used as a measure of retinal function.

While ERG measures overall retinal function, a variation called “multifocal ERG” allows for the measurement of functional response from different locations in the retina. This is accomplished by stimulating each visual field location with a stimulus sequence that is uncorrelated with the stimulus sequences used for the other locations. Retinal activity is recorded as in the usual ERG technique, and then mathematical algorithms are used to extract the distinct response for each visual field location.

A person of skill in the art may choose to measure photopic response, scotopic response, or any other assessment of retinal function, with or without the multifocal technique, to validate the therapeutic benefits of the method of the present invention.

As used herein, the term “optical coherence tomography” (OCT) refers to a noninvasive imaging technology used to obtain high resolution cross-sectional images of a retina. OCT is a standard method used in clinical assessment and treatment of retinal diseases. OCT may be used to assess cellular organization, photoreceptor integrity, macular degeneration, and other visible criteria in retinal health. Variations of OCT using modified imaging setups have been developed and may be selected by a person of skill in the art, including, for example, time domain OCT, frequency domain OCT, spectral domain OCT (SD-OCT), spatially encoded frequency domain OCT, time encoded frequency domain OCT, full-field OCT, or line-field confocal OCT. In some exemplary embodiments, retinal structure is assessed using SD-OCT.

As used herein, the term “treat” or “treatment” refers to a therapeutic measure that reverses, stabilizes or eliminates an undesired disease or disorder (e.g., retinoschisis), for example, by causing the regression, stabilization or elimination of one or more symptoms or indicia of such disease or disorder by any clinically measurable degree. For example, with regard to retinoschisis, treatment may cause a reduction in or maintenance of splitting of the retinal layers, as measured for example using OCT, or measured functionally for example using ERG.

In addition to the treatment of XLRS in human patients, it may be desirable to use the method of the invention to treat models of XLRS in non-human animals. A non-human animal used as a model of XLRS may include, for example, a fish, an amphibian, a reptile, a mammal, or a bird. In some embodiments, a non-human animal used as a model of XLRS is a mammal. In some embodiments, a non-human animal used as a model of XLRS may be a primate, a goat, a sheep, a pig, a dog, a cow, or a rodent. In some embodiments, a non-human animal used as a model of XLRS is a rat or a mouse. In some exemplary embodiments, a non-human animal used as a model of XLRS is a Rs1 KO, C59S, or R141C mutant mouse, as described in International Patent Application Publication WO 2018/157058 A1.

It is understood that the present invention is not limited to any of the aforesaid missense mutation(s), animal model(s), vector(s), promoter(s), titer(s), volume(s), amount(s), functional retinal assessment(s), or structural retinal assessment(s), and any missense mutation(s), animal model(s), vector(s), promoter(s), titer(s), volume(s), amount(s), functional retinal assessment(s), or structural retinal assessment(s) can be selected by any suitable means.

The present invention will be more fully understood by reference to the following Examples. They should not, however, be construed as limiting the scope of the invention.

### EXAMPLES

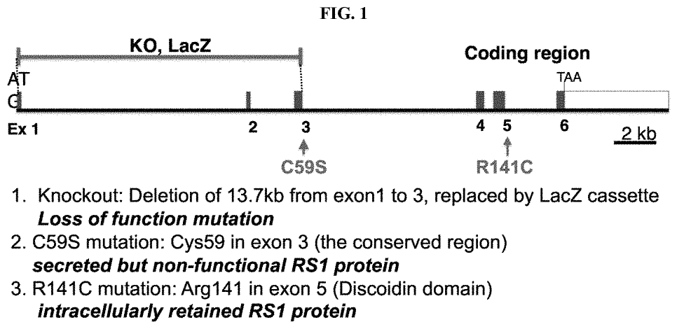

Male pups from three mouse models of XLRS (as shown in FIG. 1) were used to demonstrate rescue of retinal function and structure by gene supplementation therapy. Subretinal injection of 1 μL of AAV2 7m8-Rho-hRS1 at a titer of 3×10¹³ vg/mL was administered in the right eyes at postnatal day 21. The left eyes were used as untreated controls.

In vivo retinal structural restoration was evaluated with spectral domain optical coherence tomography (OCT). Functional rescue was evaluated with dark-adapted electroretinography (ERG). Evaluations were conducted at 2 and 4 months post-injection. Ex vivo retinal tissues harvested at 4 months post-injection were flat mounted for immunohistochemistry (IHC) using anti-RS1 and anti-arrestin antibodies to assess hRS1 expression and local photoreceptor protection. A subset of retinal tissues from each group were examined using western blot (WB) to confirm hRS1 expression.

RS1 mutant mouse models. Rs1 mutant mouse lines were generated for this study using Regeneron's VelociGene technology and described in a previous publication (Liu et al., 2019). Briefly, an Rs1 deficient line was created by deletion of 13.7 kb from exon 1 to 3, which was replaced by LacZ cassette generating a loss of function mutation. In addition, two knock-in lines were generated carrying nucleotide 175T→A (missense mutation C59S) in exon 3 (the conserved region) resulting in secreted but non-functional RS1 protein, or 421C→T (missense mutation R141C) in exon 5 (Discoidin domain) producing intracellularly retained RS1 protein, to mimic known human RS1 mutations. All the models show an early onset and severe phenotype of retinoschisis, including disruption of retinal layers and reduction of ERG b-wave, demonstrating that they are effective models of the XLRS disease state for the development of potential therapeutics.

Intravitreal or subretinal injection of the AAV2 7m8 vector containing the human RS1 cDNA under the control of the human Rhodopsin promoter (Rho). The full-length human RS1 was cloned into the pAAV plasmid containing AAV-inverted terminal repeats and a human Rho promoter which drives RS1 expression in photoreceptors. The titers of the vectors were adjusted to 3×10¹³ vector genomes (vg)/mL. Male pups from the mouse models were used for the intravitreal (IVT) or subretinal injection of 1 μL of AAV2 7m8-Rho-hRS1 on postnatal day 21 in the right eyes, and the left eyes were used as no treatment controls. The animals were housed under standard laboratory conditions (22±2° C., 60±10% relative humidity, and a 12-hour light-dark cycle) and had free access to food and water throughout the experiment. The conditions of housing and experiments were in accordance with the Association for Research in Vision and Ophthalmology Statement for the Use of Animals in Ophthalmic and Vision Research, and with the protocols approved by the Regeneron institutional animal care and use committee (IACUC).

Spectral-domain optical coherence tomography (SD-OCT). Mice were anesthetized with sodium pentobarbital (68 mg/kg) or ketamine and xylazine (120 mg/kg and 5 mg/kg, respectively). Pupils were dilated with 1 μL eyedrops comprised of 0.5% tropicamide (Alcorn Inc.; Lake Forest, Ill.) and 0.5% phenylephrine HCl, or 1% tropicamide. The corneal surface was anesthetized with a single application of 10 μL of 0.5% proparacaine. A contact lens (diameter 3.2 cm) was used during image acquisition.

SD-OCT images of the retina were collected along the horizontal and vertical meridians centered on the optic disk. Each set of orthogonal radial scans (1000 A-scans per B-scan by 10-15 frames) was converted to AVI files and exported to ImageJ with an axial scale of 1 μm/pixel. In ImageJ, each set of B-scans was co-registered and averaged using StackReg/TurboReg plug-ins. The field of view for each image was 0.464 mm (depth) by 1.4 mm (width). The area of schisis was measured from along the horizontal meridian by outlining the inner retina in ImageJ, with reference to three retinal quadrants (center, superior nasal, and superior temporal to the optic nerve head) of each eye at different time points. ONL thickness was measured halfway from the optic disk to the edge of the image using the straight line tool. SD-OCT images were obtained from both eyes in the nasal and temporal cardinal directions from the optic nerve head.

Electroretinography (ERG). Animals were dark-adapted overnight in a temperature- and humidity-monitored cabinet and then were anesthetized with ketamine/xylazine (80 mg/kg and 16 mg/kg, or 120 mg/kg and 5 mg/kg) diluted in 0.9% saline. Eye drops were used for pupil dilation (1% tropicamide, 2.5% phenylephrine HCl and 1% cyclopentolate, or 1% tropicamide) and to anesthetize the corneal surface (0.5% or 1% proparacaine HCl).

ERG measurements were made using the Colordome Lab Cradle by Diagnosys LLC. Mice were placed onto the testing platform, which contained a heated pad (37° C.) to maintain body temperature. Gold electrodes shaped into a circle with similar size to a mouse cornea were placed on the corneas of both eyes and served as active electrodes. A thin layer of tear gel was applied over the eyes after electrode placement. Alternatively, ERGs were obtained from the corneal surface using a stainless steel wire that was coiled at the end and wetted with a small drop of 1% carboxymethylcellulose. A subcutaneous needle electrode inserted on the head between the eyes was used as a reference electrode, and a subcutaneous needle electrode inserted on the body near the base of the tail served as the ground electrode. All operations including anesthetizing animals, animal positioning and electrode placement were performed under dim red light, to preserve dark adaptation.

ERG testing was done over the course of several days. At the beginning of each testing day, a WT control mouse was first recorded to ensure the system was working optimally. Following completion of ERG testing, scans were exported and then imported into an in-house ERG Analyzer program which allows for easy visualization of scan results. The program automatically detects b-wave (as the highest electrical amplitude in the graph) and a-wave (as the minimum occurring before the identified b-wave). With some scans, however, manual adjustments needed to be made, which were recorded. A- and b-wave amplitudes for each step in the protocol were recorded in an excel sheet.

Flash stimuli were presented in an LKC (Gaithersburg, Md.) ganzfeld, first to the dark-adapted eye (−3.6 to 2.1 log cd s/m²) and then superimposed upon a steady 20 cd/m² background field following a five minute light adaptation period (−0.8 to 2.1 log cd s/m²). ERGs were amplified (0.03-1000 Hz), averaged and then stored using an LKC UTAS E-3000 signal averaging system.

The amplitude of the a-wave was measured at 8 ms after flash onset from the pre-stimulus baseline. The leading edge of the a-wave to a 1.4 log cd s/m² flash was measured using the equation

P3(i,t)=(1−exp(−iA(t−td)²))R_{mP3}

where R_{mP3} is the maximum response amplitude, A is a measure of sensitivity and td is the delay in phototransduction.

The amplitude of the b-wave was measured from the a-wave trough to the b-wave peak. The function relating b-wave amplitude to flash luminance has two limbs. The first limb of this function is fitted using the Naka-Rushton equation:

$$\frac{R}{{R}_{max}} = L / ( L + K )$$

where R is the amplitude of the a- or b-wave, Rₘₐₓ is the maximum amplitude of the a- or b-wave, L is the flash energy (log cd s/m²), and K is the flash energy that elicits an amplitude of half Rₘₐₓ (half-saturation coefficient).

Statistical analysis. All statistical analysis was performed in GraphPad Prism v8 software. For dark-adapted b-wave analysis, the b-wave amplitudes at each stimulus intensity were graphed for each mouse, to observe whether there was any difference in retinal function between injected (OD) and un-injected (OS) eyes, and to see how the function in Rs1 KO mice compared to WT controls. The means of b-wave amplitude at several intensities among the various groups were compared using a two-way ANOVA. Additionally, area under the curve (AUC) was calculated for the b-wave amplitude at several intensities for each mouse. The AUC in the un-injected OS eye was subtracted from the AUC in the injected OD eye, in an effort to quantify the magnitude of the difference between them, and therefore, the magnitude of the treatment effect. AUCs were then compared between the various groups using an ordinary one-way ANOVA.

Immunohistochemistry (IHC). Animals were euthanized 4 months post-injection by CO₂ inhalation. Eyes were enucleated and fixed with 4% PFA (Electron Microscopy Sciences) in 1×PBS (ThermoFisher Sci.) for three hours at 4° C. After three washes with 1×PBS, eyes were dissected under a dissecting microscope. The anterior segment and lens were removed, and the rest of the eye (eyecup) was incubated in 30% sucrose at 4° C. overnight. Eyecups were then embedded in TISSUE-TEK® O.C.T. Compound (VWR) and snap frozen on dry ice. 10 μm cryostat sections were prepared on SUPERFROST® Plus Micro Slide used for immunofluorescent staining. Sections on slides were encircled with Liquid Blocker Super Pap Pen (Electron Microscopy Sciences) and air dried for 30 minutes at room temperature. Blocking solution was prepared as 5% normal goat serum (VectorLabs), 1% bovine serum albumin (Sigma-Aldrich) and 0.3% Triton-X 100 (Sigma-Aldrich) in 1×PBS. Wash solution was prepared as 0.1% Tween 20 (Amresco) in 1×PBS. Slides were placed in a staining container with a black lid, and washed three times with 1×PBS to remove O.C.T. compound. Blocking solution was added to slides and remained for one hour at room temperature. After removal of blocking solution, primary antibodies were diluted in blocking solution and applied to sections overnight at 4° C. On the second day, slides were washed three times with wash solution. Fluorophore-conjugated secondary antibodies were diluted at 1:1000 in 1×PBS and applied on sections for one hour at room temperature (in the dark to avoid photobleaching). Slides were washed three times with 1×PBS and coverslipped with ProLong Gold Antifade Mountant with DAPI (ThermoFisher Sci.).

Alternatively, RS1 mutant and age-matched WT control eyes were fixed with 4% PFA for one hour at room temperature, and retinas were dissected and prepared for flat mounts. To stain for RS1 and cone arrestin, retinal flat mounts were blocked for one hour at room temperature in the blocking buffer (5% goat serum, 1% bovine serum albumin and 0.5% Triton-X 100). Following blocking, retinas were incubated in the primary antibodies, diluted in blocking buffer, at 4° C. overnight. Primary antibodies included mouse anti-human RS1 (1:100, Abnova, Cat #H00006247-B01P, lot: GB291) conjugated to Alexa Fluor 647 (ThermoFisher Scientific, Cat #C20029) with a site-specific antibody labelling kit (ThermoFisher Scientific, Cat #S10900) and rabbit anti-mouse cone arrestin (1:500, EMD Millipore, Cat #AB15282, lot: 3475937). After several washes with the wash buffer (0.5% Tween-20 in 1×PBS), retinas were then incubated with the secondary antibody solution, containing goat anti-rabbit Alexa Fluor 488 (1:1000, ThermoFisher Scientific, Cat #A11008, Lot: 1797971) diluted in 1×PBS. Following a final round of washes in the wash buffer, retinal flat mounts were mounted on glass slides and mounted with Prolong Glass anti-fade mounting medium (ThermoFisher Scientific, Cat #P36980), with the photoreceptor layer facing the coverslip.

Images of the entire retinal flat mounts with multiple z-stacks were taken with the Leica Thunder Imager microscope (model: Leica DM6B) using a 10× objective lens and the GFP (Alexa Fluor 488) and Cy5 (Alexa Fluor 647) filters. For Alexa Fluor 488 detection, fluorescence intensity was set to 55%, exposure to 30 ms, and gain to low noise and high well. For Alexa Fluor 647 detection, fluorescence intensity was set to 30%, exposure to 25 ms and gain to low noise and high well. Following image acquisition, images were processed with the native Las X software (version: 3.7.0.20979). Computational clearing was performed using the Small Volume Computational Clearing (SVCC) algorithm. Images were merged using the mosaic merge function, without blending. Finally, maximum intensity projections (MW) were generated to produce a 2D in-focus image. The signal in both channels was scaled (0-2500 for Alexa Fluor 488m, 0-1000 for Alexa Fluor 647), and these thresholds were kept consistent between all images. The final processed and scaled images exported in TIF format using lossless compression.

Immunocytochemistry. Stable RS1-WT, RS1-059S and RS1-R141C cells were grown on glass coverslips. The cells were transiently transfected by RS1 WT with His tag plasmid by Lipofectamine 3000 methods for 24 hours. The cells were fixed with 4% PFA at room temperature for 30 minutes, and then incubated with 0.5% Triton 100 in PBS for 20 minutes. The cell preparations were blocked for 1 hour in PBS containing 1% BSA, 5% GS, and 0.3% Triton 100. Primary antibodies were diluted in the same solution and applied for 2 hours, followed by incubation for 1 hour in the appropriate secondary antibody. After staining, the cells were mounted on a slide and fluorescent signals were visualized and captured using an open-field Nikon Eclipse Ti-E microscope.

Western blot and co-immunoprecipitation (co-IP). Total retinal protein (10-50 μg), total cell lysate protein, or concentrated supernatants (Amicon centrifugal filters 3K, UFC500396, Millipore) were loaded onto 4-12% Bis-Tris gels in sample buffer (Nupage LDS sample buffer, Thermo) containing 4% SDS for protein separation and then transferred to nitrocellulose membrane (0.45 μm pore size, Invitrogen) followed by blocking with SuperBlock T20 (TBS, Thermo) for one hour at room temperature. Blots were incubated with anti-RS1 antibody (Novus Biologicals USA, 1:4000 or 1:5000) for two hours at room temperature or overnight at 4° C. After incubation with anti-RS1 antibody, HRP conjugated anti-mouse polyclonal antibody (Cell Signaling) was added at 1:5000 for one hour at room temperature.

For co-IP, 3 μg of mouse anti-His (MA1-21315, Thermo) or mouse anti-myc (MA1-21316, Thermo) monoclonal antibodies were incubated with Dynabeads™ Protein G (10003D, Thermo) and rotated at room temperature for 30 minutes. The beads were washed with 0.2% Tween 20 in PBS twice, then incubated with the supernatants at 4° C. for 3 hours. Elution buffer was added after three washes, and his and myc expression were evaluated using rabbit anti-His (2365s, Cell Signaling) and rabbit anti-myc (2278s, Cell Signaling) antibodies.

Protein bands were visualized and imaged using SuperSignal West Pico chemiluminescence (Thermo) by C-Digit Blot Scanner (Li-Cor).

RNAscope. The expression pattern of hRS1 mRNA in treated eyes was determined by in situ hybridization using RNASCOPE® according to the manufacturer's specifications (Advanced Cell Diagnostics). Briefly, formalin or 4% paraformaldehyde (PFA)-fixed and paraffin or O.C.T. embedded mouse eye cups were cut into 5 to 10 μm sections and mounted on SUPERFROST® Plus glass slides. The procedure began with 10 minute Pretreat 1 (ACD, 320037) at room temperature, followed by 20-minute boiling in Pretreat 2 (ACD, 320043) with Oster Steamer (IHC World, LLC, Model 5709) and Pretreat 3 (ACD, 320037) for 30 minutes at 40° C. in a HybEZ Oven (ACD, 310010). An additional DNase treatment step was included to reduce potential background from probe hybridization with chromosomal DNA. Slides were then washed three times with water, and a solution of DNase I (AM2224, Ambion) was added to the eye tissue for a 30 minute incubation at 40° C. Slides were then washed five times with water, hybridized with RNASCOPE® probes for two hours at 40° C. and the remainder of the manufacturer's assay protocol was implemented (ACD, 322360) from step Amplified 1 to step Amplified 6. The slides were washed twice (two minutes each at room temperature) using RNASCOPE® wash buffer (ACD, 310091). After the Amplified steps, signal was detected by incubation with Red working solution (1:60 ratio of Red B to Red A) at room temperature for 10 minutes in the absence of light, followed by washing the slides in water several times and viewing under a microscope.

### Example 1. Co-Expression of WT RS1 Induces Secretion and Octamerization of Disease Mutant RS1

In order to explore the potential for co-assembly of wildtype (WT) RS1 protein with either secreted or non-secreted disease variants, cell lines stably expressing WT RS1 (His-tagged), C59S (myc-tagged) or R141C (myc-tagged) were constructed. Transfection of wildtype RS1-expressing cells with mutant RS1 led to co-assembly and secretion of wildtype/mutant RS1 heteromers.

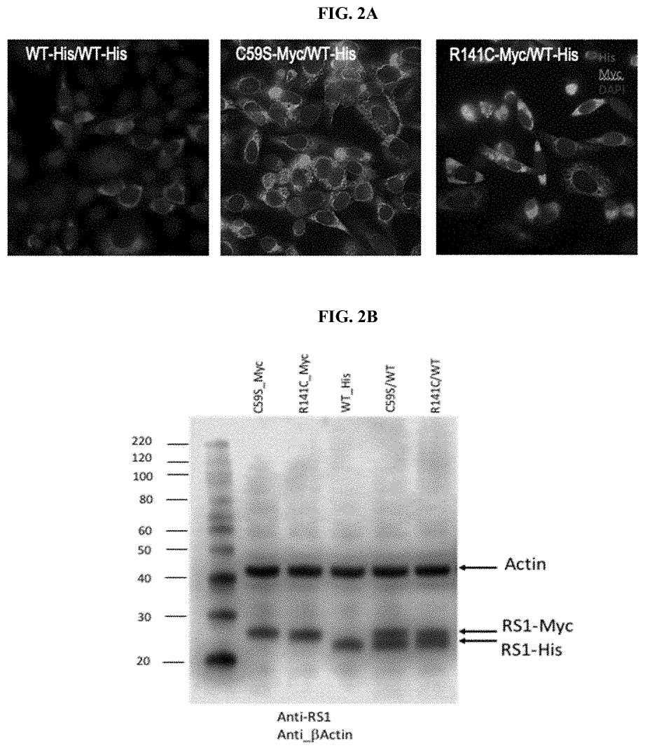

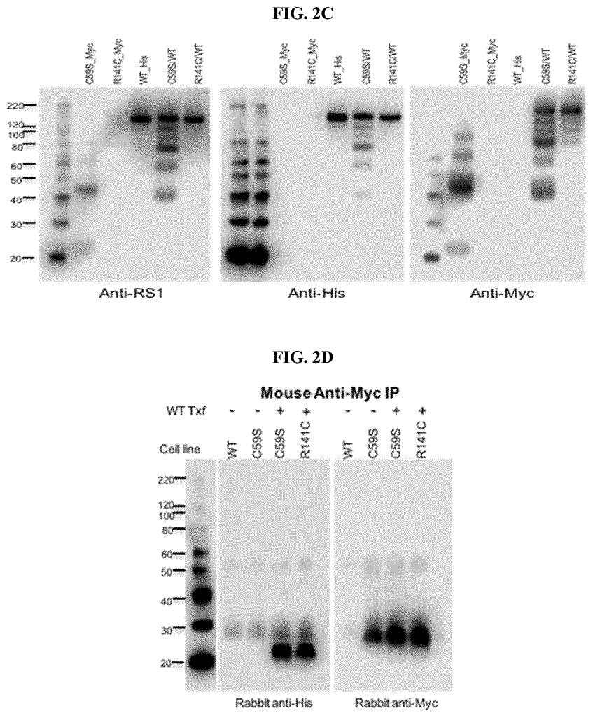

RS1 protein was produced by all the cell lines, as shown by western blotting of reducing gels of the cell lysate, as shown in FIG. 2B. As expected, non-reducing western blots demonstrated that WT was secreted exclusively as an octamer, C59S predominantly as a dimer (with minor amounts of monomer and some higher order species but no octamer detected), and R141C was not secreted, as shown in FIG. 2C.

Each of these cell lines was additionally co-transfected with plasmids encoding WT RS1-His. Immunocytochemistry indicated many cells had co-expression of His- and myc-tagged proteins, as shown in FIG. 2A. Co-localization of WT and mutant RS1 was detected with anti-His (red) and anti-myc (green) antibodies, with magnification 100×.

In the C59S cells transfected with WT RS1, non-reducing western blots indicated that secreted C59S myc-tagged RS1 was now present to a greater extent in higher size complexes, including the octamer band. His-tagged WT RS1 in these co-transfected cells was present in smaller bands in addition to the octamer, including in the dimer, as shown in FIG. 2C. These results indicated that WT and C59S RS1 interacted with each other in the co-transfection: WT RS1 tended to form heteromers with C59S RS1, leading to generation of intermediate species in addition to the octamer observed in WT RS1 single transfection or the predominant dimer in C59S Rs1 single transfection. In the R141C cells transfected with WT RS1, the myc-tagged variant was now present extracellularly in the octamer position, as shown in FIG. 2C. This is strong evidence that the presence of WT RS1 enables assembly and secretion of disease variants, presumably via co-assembly into multimers with WT.

In order to confirm a direct interaction of WT RS1 with mutant subunits, co-immunoprecipitation experiments were performed in Cho-K cells, as shown in FIG. 2D. Antibody against myc did not pull down His-tagged WT RS1 from the medium of cells expressing only the WT RS1, but did pull down His-tagged WT protein from the cells expressing the variants that had been co-transfected with WT RS1. This indicates a direct interaction between the WT and co-expressed variant proteins.

### Example 2. Intravitreal Administration of AAV2-7m8 Rho-hRS1 does not Provide Structural or Functional Recovery in Rs1 Deficient Mice

Several reports of disease mitigation in Rs1 KO mice by IVT administration of AAV viral vectors have been published (Zeng et al., 2004; Kjellstrom et al., 2007; Park et al., 2009, Seok et al., 2005; Janssen et al., 2008; Molday et al., 2012). Based on the ability of exogenous WT RS1 to form an octamer with R141C, as shown in Example 1, an AAV2-7m8 vector that delivers WT RS1 protein under control of the rhodopsin (Rho) promoter was created and delivered via IVT injection (3e13 vg/mL x 1 μL) into R141C mice as well as Rs1 KO mice.

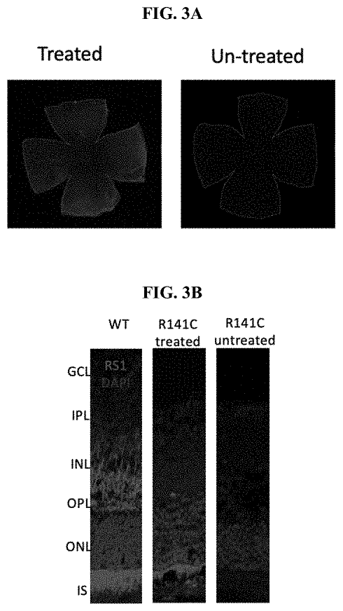

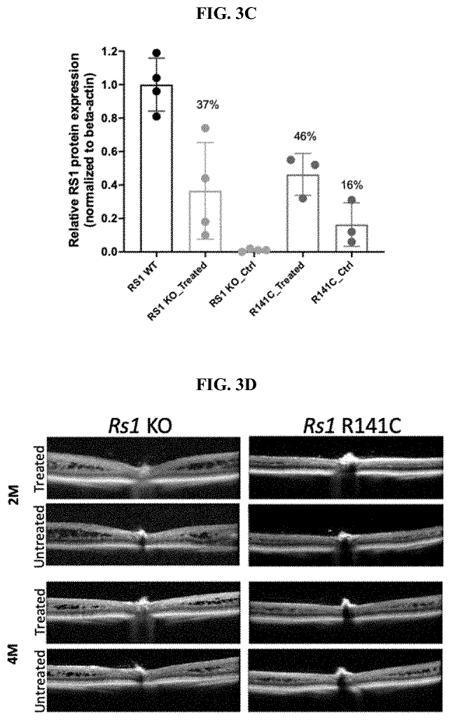

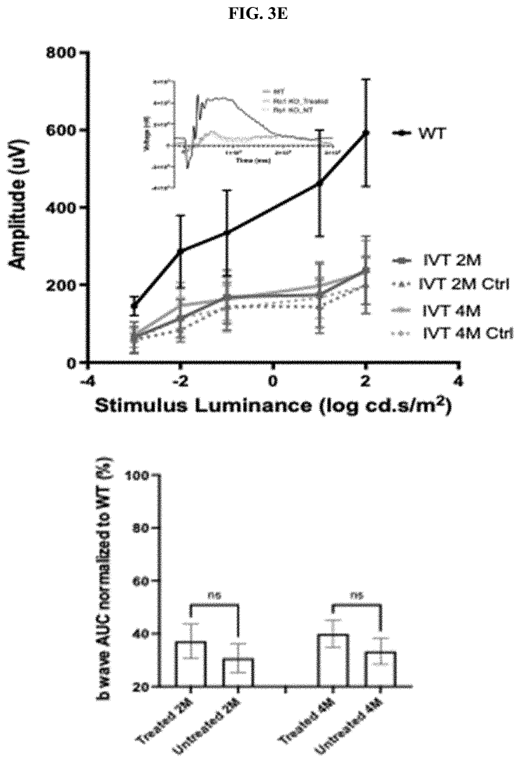

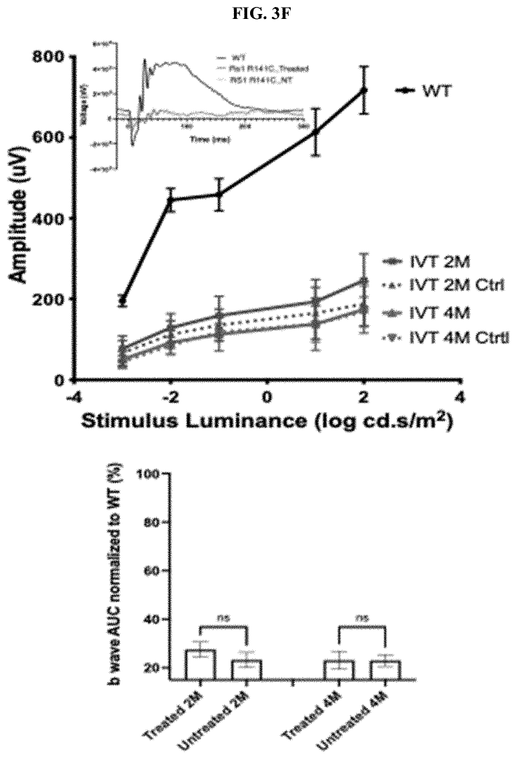

IHC in retinal flat mounts of Rs1 KO mice showed significant and homogeneous RS1 positive signal in the treated eye, as shown in FIG. 3A. RS1 expression was further assessed using IHC in retinal cryosections of R141C mice 2 months after AAV 7m8 RS1 IVT injection. RS1 was expressed with some intensity in ONL and INL, but significantly lower signal strength in photoreceptor IS, as shown in FIG. 3B.

Automated western blot under reducing conditions showed that RS1 expression levels 6 months after IVT injection of the viral vector were about 37% and 46%, respectively, in the retinal tissues of Rs1 KO and R141C mice compared to Rs1 WT mice, as shown in FIG. 3C. Surprisingly, despite the presence of RS1 protein expression, neither retinal structural protection nor retinal functional rescue was observed. Retinal structural protection was measured using OCT imaging 2 or 4 months after IVT injection of 7m8-Rho-hRS1, as shown in FIG. 3D. Retinal functional rescue was measured by ERG in Rs1 KO and R141C mice, as shown in FIG. 3E and FIG. 3F respectively.

### Example 3. Subretinal Administration of AAV2-7m8 Rho-hRS1 Provides Structural and Functional Recovery in Rs1 Deficient Mice

In order to model the treatment of XLRS caused by missense mutations of RS1, four mouse models were used: wildtype mice, and RS1 KO, C59S, and R141C mice, as described in International Patent Application Publication WO 2018/157058 A1. A modified adeno-associated virus, AAV2.7m8, was used as viral vector for gene therapy treatment (Dalkara et al., 2013, Sci. Transl. Med., 5(189):189a76). The therapeutic transgene comprised wildtype human RS1, operably linked to a rhodopsin kinase promoter.

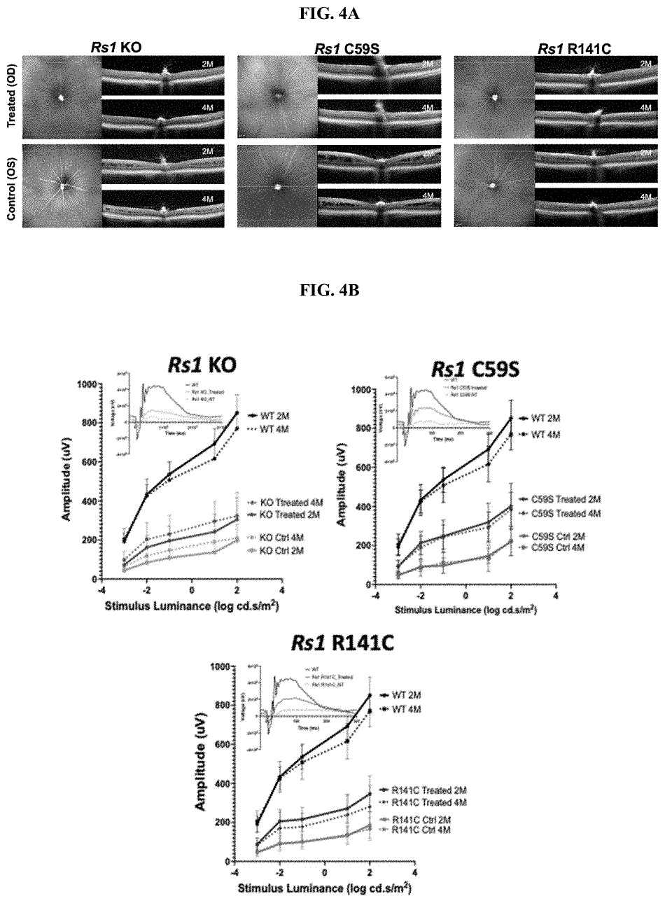

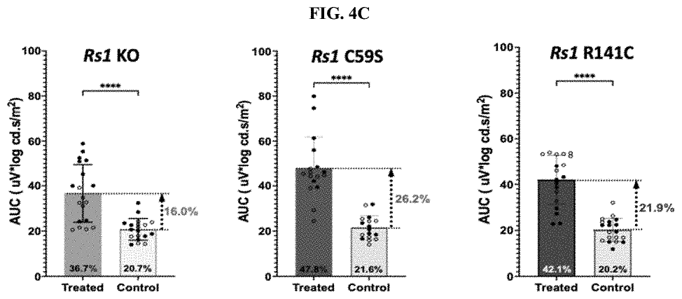

Subretinal injections of 3×10¹⁰ vg/eye of 7m8-Rho-RS1 were administered to Rs1 KO, C59S and R141C mice on postnatal day 21 in the right eyes, while left eyes were used as untreated controls. OCT was used to assess the structural rescue of retinal layers 2 months and 4 months post-injection. Gene supplementation therapy with subretinal administration successfully rescued retinal structure 2 months and 4 months post-injection in each of the mouse models of XLRS, as shown in FIG. 4A, indicating that long-lasting therapy had been achieved. In contrast, control eyes of the same animals showed splitting of the retinal layers, characteristic of untreated retinoschisis.

Treated mice were further tested for restoration of retinal function, in comparison both to the untreated eye of the same animal and to wildtype mice. Eyes were dark-adapted and then assayed using ERG, and the amplitude of b-wave responses to stimuli of varying luminance was used as a measure of retinal function. The treated eyes (OD) of mouse models of XLRS showed improved performance in dark-adapted ERG relative to untreated eyes (OS), as shown in FIG. 4B. Improvement was seen at both 2 and 4 months post-injection, and in missense mutant mice as well as in Rs1 KO mice.

Overall functional improvement can be quantified by comparing the area under the curve (AUC) of dark-adapted ERG b-wave in treated eyes versus untreated eyes, as shown in FIG. 4C. The relative retinal function of Rs1 KO, C59S, and R141C mice was 36.7%, 47.8%, and 42.1%, respectively, compared to WT animals. Functional improvement was 16.0%, 26.2%, and 21.9%, respectively, compared to untreated control eyes. Statistical analysis was conducted using a 2-tailed unpaired t test, with **** in FIG. 4C indicating p<0.0001.

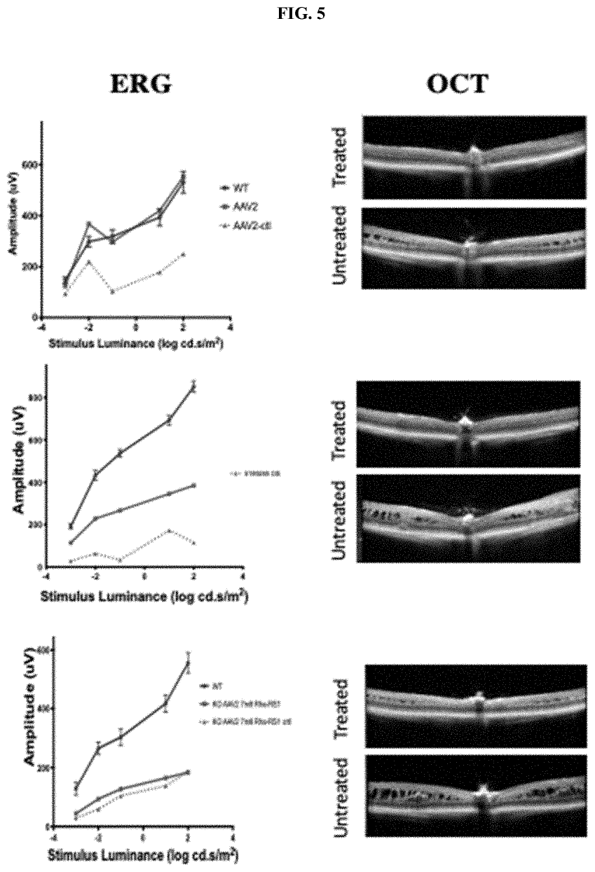

The variability in ERG amplitudes among treated animals was large, ranging from no functional preservation to nearly 100% of WT levels maintained, as shown in FIG. 5. In the eye that had little to no visual function as measured by full-field ERG, shown in the bottom example of FIG. 5, structural protection was near complete, as evidenced by much fewer cysts or retinoschisis seen in OCT. Both partially and fully functionally preserved eyes exhibited complete structural restoration, as seen in the top two examples of FIG. 5.

While there is variable measured functional rescue between the conditions, potentially due to biological variation, technical variation, or insensitivity of the ERG assay to smaller or local effects, the results of the ERG assay demonstrated that subretinal gene supplementation therapy was able to significantly rescue function in each of the three XLRS mouse models. The rescue of retinal structure and function in the missense mutants was comparable to rescue in the Rs1 KO mutant, demonstrating for the first time that gene supplementation therapy may be an effective treatment for XLRS caused by missense mutations of Rs1.

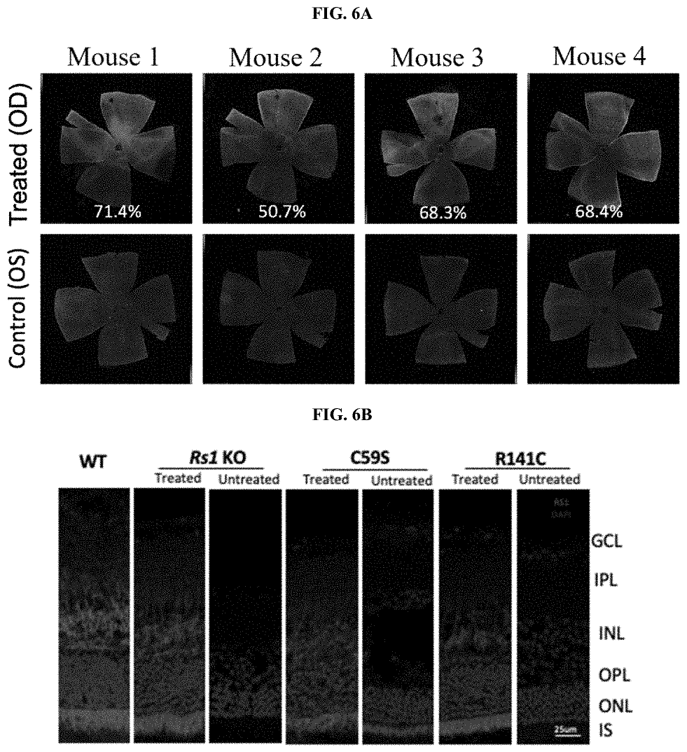

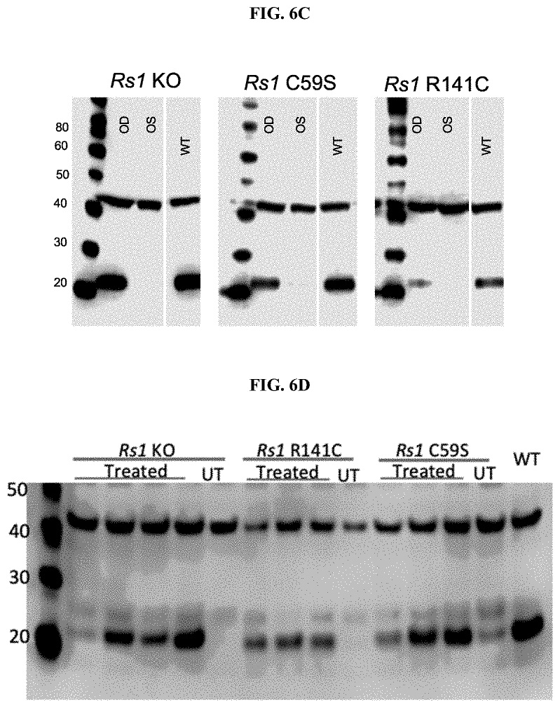

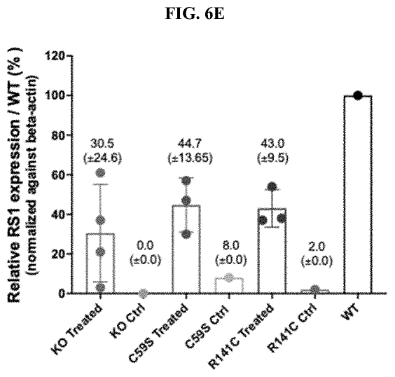

In order to further characterize the gene therapy of the present invention, retinal samples of treated mice were assayed for expression of hRS1 protein 4 months post-injection. Retinal sections from RS1 KO mice were flat mounted and analyzed using IHC, as shown in FIG. 6A. An antibody against RS1 was used to characterize expression and localization of hRS1. RS1 is shown in red and cone arrestin is shown in green. The retinal areas covered by hRS1 positive signal comprised 50-70% of the total area, showing widespread expression of the transgene 4 months after subretinal injection. hRS1 expression was further assessed in retinal cryosections, as shown in FIG. 6B.

Retinal samples from Rs1 KO, C59S and R141C mice were additionally assessed by western blot under reducing conditions and probed with an anti-RS1 antibody, as shown in FIG. 6C. The presence of a band at the correct size for monomeric RS1 was compared between treated eyes (OD), untreated eyes (OS), and wildtype (WT) mice. Monomeric RS1 was present in treated but not untreated eyes at a level comparable to wildtype expression, demonstrating robust expression of the transgene in targeted retinas using the gene supplementation therapy of the present invention 4 months after subretinal injection.

Additional western blot analysis under reducing conditions, 6 months after subretinal injection, is shown in FIG. 6D. The western blot under reducing conditions showed clear monomeric RS1 bands from retinal samples of the treated eye 6 months after subretinal injection, but no (in KO and R141C models) or minimally visible (in the C59S model) bands from the control eye (OS), demonstrating robust expression of the transgene in targeted retinas using the gene supplementation therapy. The densitometry of the protein bands of FIG. 6D for RS1 quantification showed variable RS1 expression level in treated eyes of Rs1 KO, C59S and R141C mouse retinas (30.5% and 44.7% and 43.0% compared to RS1 in WT animal retina, respectively), as shown in FIG. 6E. The considerable variability in protein expression was potentially due to biological variation or technical variation.

### Example 4. Comparison of Intravitreal and Subretinal Administration

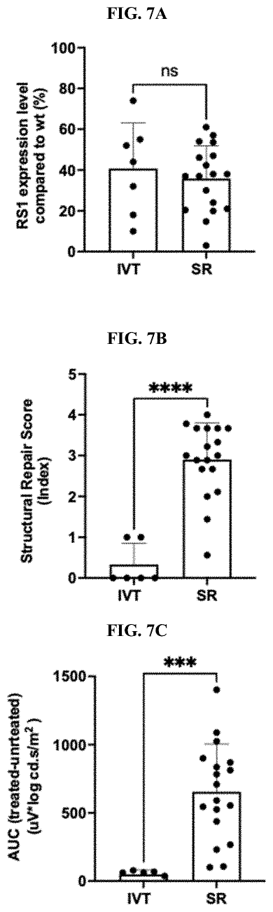

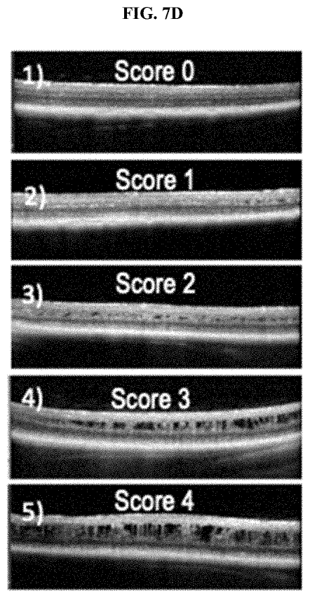

Mice treated with gene supplementation by subretinal injection were compared to mice treated by intravitreal injection. The overall relative amount of RS1 expression compared to WT mice in eyes treated by subretinal delivery of the transgene was comparable to those eyes treated by IVT injection, as shown in FIG. 7A. The IVT samples had a score of 40.7%±22.3% while the subretinal samples had a score of 35.8%±16.2%. However, there were significant differences in RS1 protein spatial distribution (data not shown), structural restoration (FIG. 7B), and retinal functional rescue (FIG. 7C).

Structural restoration was represented by the Schisis Repair Score (4 minus the Schisis Score). The IVT samples had a score of 0.6±0.8, while the subretinal samples had a score of 2.9±0.9. Functional rescue was indicated by AUC of scotopic ERG b-wave, using the treated eye minus the untreated eye. The IVT samples had a score of 48.33±33.89 uV*log cd·s/m², while the subretinal samples had a score of 654.5±350.9 uV*log cd·s/m². Statistical analysis was conducted using an unpaired two-tailed t test for protein expression comparison and dark-adapted ERG b-wave AUC, and a Mann-Whitney U test for retinal structural repair. NS indicates not significant, *** indicates a p value less than 0.01, and **** indicates a p value less than 0.001.

FIG. 7E illustrates scoring of retinal schisis. Scoring of retinal schisis was conducted independently by three readers from OCT images by overall ranking of the cavities from three different areas containing 31 images each. A score of 0 indicated that no cavities were observed. A score of 1 indicated that 1 to 5 cavities were observed on at least one individual image. A score of 2 indicated that greater than 5 cavities were observed on at least one individual image, but the cavities were not fused. A score of 3 indicated that there were fused cavities on at least one individual image. A score of 4 indicated that there were fused cavities on at least one individual image and the retina was stretched. The averaged Schisis Score for each eye by three readers was obtained. The Schisis Repair Score was calculated by subtracting the Schisis Score from 4.

### Example 5. Additional Structural and Functional Protection and Benefit for Cone Cells Using Subretinal Administration

The structural and functional measures of rescue and transgene expression after gene supplementation therapy described in the above examples provide a measurement of overall retinal recovery. Additional experiments were conducted to assess local rescue and transgene expression effects after subretinal injection that may not be detected by broader methods.

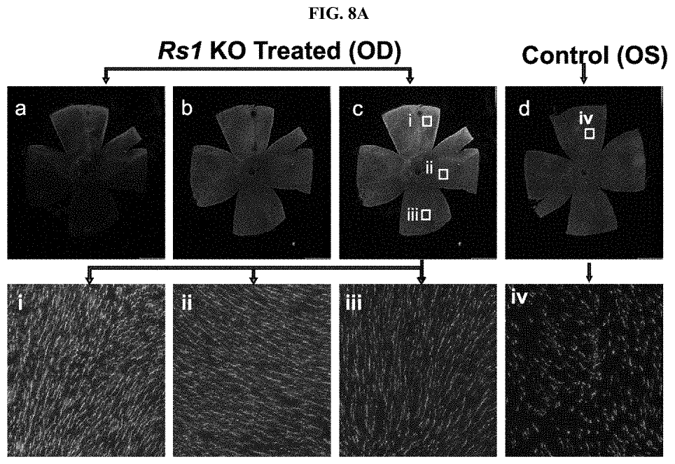

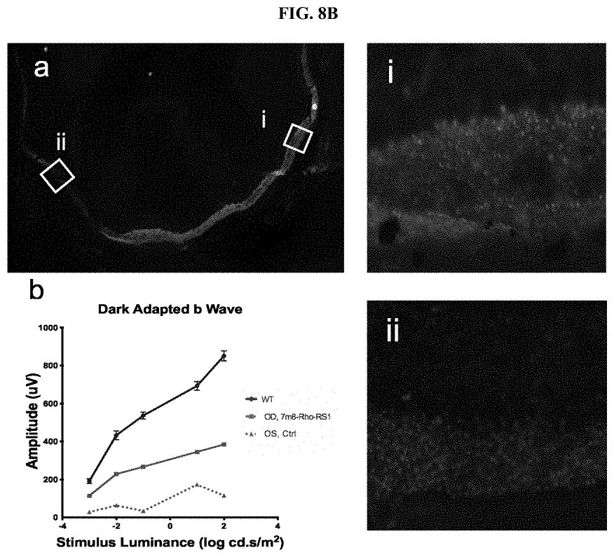

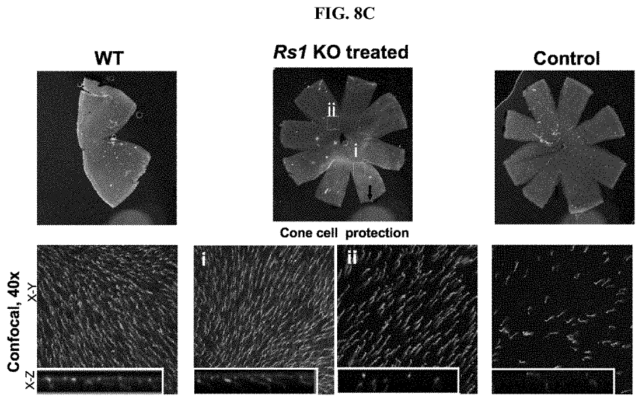

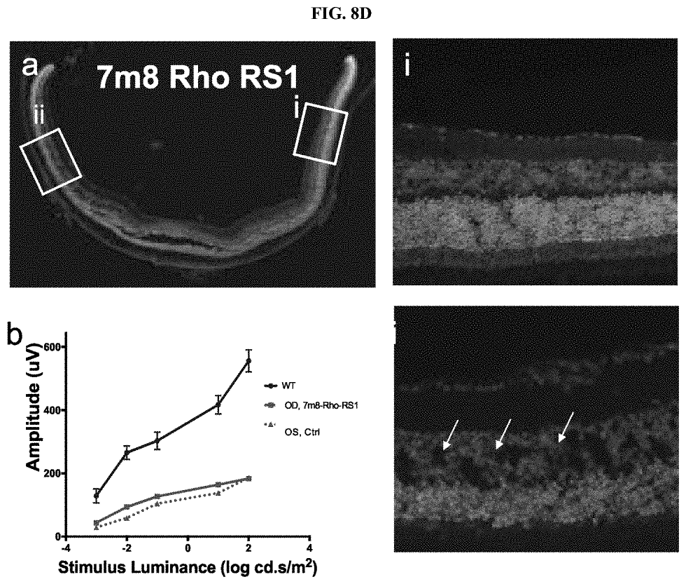

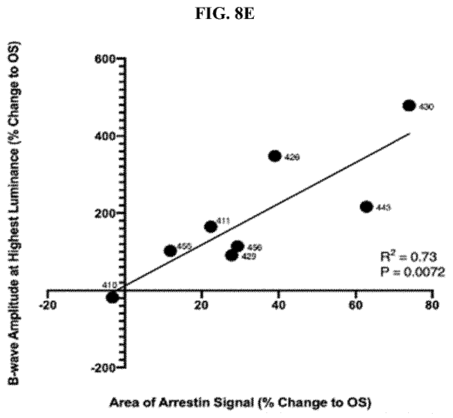

Retinal sections of treated Rs1 KO mice 4 months after subretinal injection were flat mounted and analyzed using IHC, as shown in FIG. 8A. An antibody against hRS1 was used to characterize expression and localization of hRS1, shown in red, while an antibody against cone arrestin was used as a marker of cone cells, shown in green. Cone cell protection was seen where hRS1 was present (i and ii), but also where it was not (iii), in the treated eyes. In contrast, the untreated eyes showed a significant relative decrease in cone cells (d and iv). This result demonstrates that the gene therapy of the present invention provided significant protection of cone cells, not only in areas expressing hRS1, but throughout the retina. While the dark-adapted ERG results described in Examples 3 and 4 demonstrated rescue of rod-pathway retinal function, the protection of cone cells seen by IHC suggests that cone-pathway retinal function may also be rescued using the gene therapy of the present invention.

Expression of hRS1 mRNA in the treated retina of a Rs1 KO mouse 4 months post-injection was also assessed, using RNAscope, as shown in FIG. 8B. hRS1 mRNA is shown in red, and nuclei stained by DAPI are shown in blue. Retinal structure was intact both in regions expressing hRS1 mRNA (i) and where hRS1 mRNA was absent (ii), demonstrating again that functional hRS1, which is secreted from the cell, can provide therapeutic benefit throughout the retina even without universal expression. The dark-adapted ERG responses for the treated and untreated eye of this mouse are shown in panel (b), demonstrating that the structural rescue of this retina corresponded to a broad functional rescue.

As seen earlier in FIG. 4C, the degree of functional rescue of the retina after gene therapy as measured by dark-adapted ERG varied between animals. However, broad measurements of overall retinal function may obscure local rescue of regions expressing the hRS1 transgene. Using IHC it was discovered that treated retinas that did not demonstrate overall functional rescue using dark-adapted ERG still showed local structural rescue and cone cell protection, as shown in FIG. 8C, FIG. 8D, and FIG. 5. FIG. 8C shows the expression of RS1, shown in red, and cone arrestin, shown in green, in a treated retina from an Rs1 KO mouse 4 months post-injection, compared to the untreated retina of the same mouse and a wildtype mouse. The treated retina showed RS1 expression and protection of cone cells comparable to the wildtype mouse, demonstrating a structural rescue of the retina, even though this retina did not demonstrate a broad functional rescue when measured by dark-adapted ERG.

FIG. 8D shows a Rs1 KO retina 4 months post-injection with RS1 expression and local structural protection (i), but with some splitting of the retinal layers in areas without RS1 expression (ii). RS1 is shown in red, and nuclei stained by DAPI are shown in blue. Panel (b) shows that the measurement of overall retinal function using dark-adapted ERG did not detect a rescue despite the clear local structural rescue seen by IHC.

Photopic ERG b-wave was assessed to determine whether there was a functional recovery correlated to cone cell structural improvement 10 months after subretinal injection, as shown in FIG. 8E. Using a linear regression, significant correlation was found between photopic ERG b-wave at the highest luminance, and the area of arrestin signal difference in treated versus untreated eyes, representing cone cell structural improvement.

Thus, the gene therapy of the present invention was shown to effect local structural rescue, widespread cone protection, and functional recovery of cone cells as indicated by photopic ERG b-wave measurements, even in retinas in which an overall functional rescue was not detectable using conventional dark-adapted ERG. The use of a more sensitive functional test may be necessary in order to fully characterize the local retinal rescue effects, for example multifocal ERG.

### Example 6. Structural and Functional Rescue is Provided by Photoreceptor-Targeted RS1 Expression

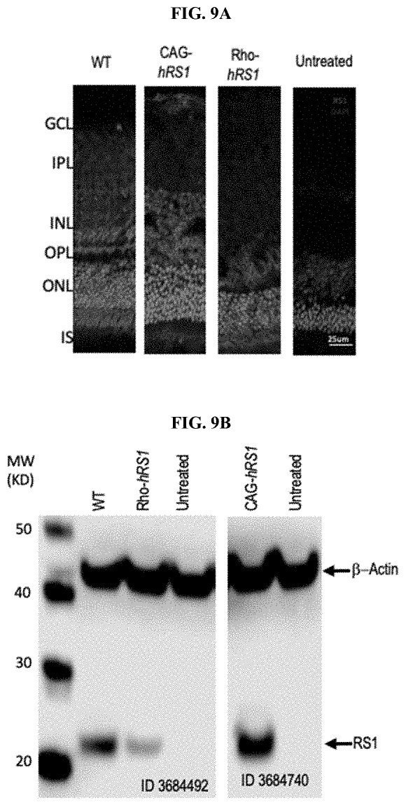

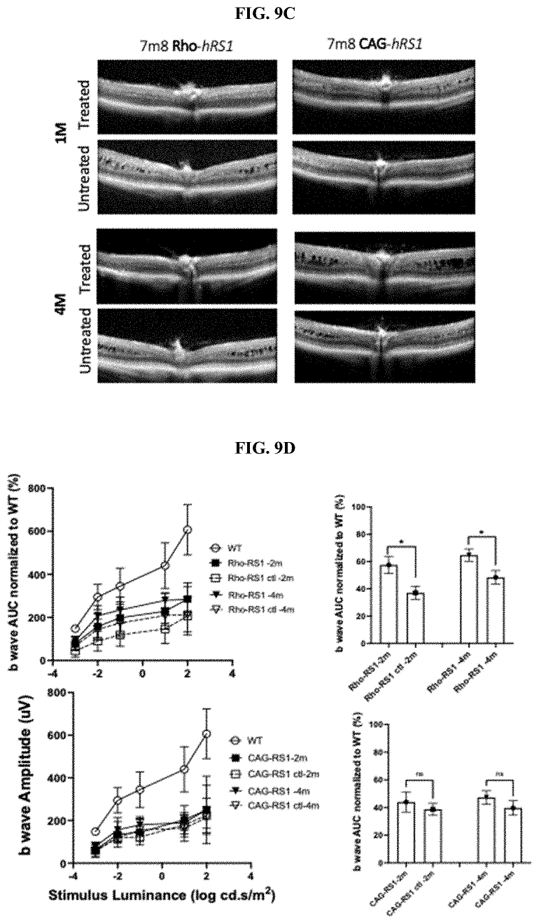

In order to assess the effectiveness of XLRS gene supplementation therapy targeted specifically to photoreceptors, compared to non-specific targeting, a vector comprising the rod-specific Rho promoter and a vector comprising the non-cell-type specific CAG promoter were compared. Mice were subretinally administered either 1 uL of 1.6e13 vg/mL 7m8 CAG-hRS1 vector, or 1 uL of 2.2e13 vg/mL 7m8 Rho-hRS1 vector. IHC of cryosections demonstrated that RS1 was distributed homogenously across the whole retina in eyes injected with CAG vector, while the majority of RS1 immune response in Rho vector-injected eyes was in the photoreceptor inner segment, as shown in FIG. 9A. Western blots in reducing conditions showed that the RS1 band intensity from CAG vector-injected eyes was much strong than in Rho vector-injected eyes, as shown in FIG. 9B. Despite this high expression level, no significant retinal structural restoration or functional restoration was observed in CAG vector-injected eyes, unlike Rho vector-injected eyes, as shown in FIGS. 9C and 9D. These results demonstrate that effective gene supplementation therapy for XLRS critically depends on choice of promoter.

As demonstrated in the above examples, gene supplementation therapy administered by intravitreal injection of 7m8 Rho-hRS1 vector yielded neither structural nor functional recovery in Rs1 KO or mutant variant mouse models, even though protein quantification showed significant hRS1 expression after the treatment injection. Surprisingly, subretinal delivery of the same vector provided only regional RS1 expression, but significant and sustained retinal structural and functional improvement. This successful treatment, comparably effective to gene therapy treatment of a Rs1 KO mouse model, suggests that gene supplementation therapy may be an effective strategy for treating patients suffering from XLRS.

Examples 3-6 illustrate that retinal structure rescue was observed 2 months through 4 months after subretinal injection of 7m8-Rho-hRS1 in all three Rs1 mutant mouse models, as demonstrated by disappearance of retinoschisis and well-organized layers on OCT imaging, compared to untreated eyes which showed worsening retinal splitting and disorganization over time. Dark-adapted ERG b-wave at 2 months post-injection demonstrated significant functional restoration in Rs1 KO, C59S and R141C mouse models respectively, relative to untreated eyes, showing for the first time that gene supplementation in Rs1 missense mutant models of XLRS was as effective of a treatment as gene replacement in a Rs1 KO model. This restoration was maintained through 4 months post-injection.

IHC in retinal flat mounts showed that 50-70% of retinal areas were covered by RS1 positive signal in treated eyes. Western blot under reducing conditions showed that retinal samples from treated eyes expressed hRS1 visible in a monomer band. Cone photoreceptor protection was observed locally where there was RS1 expression, and even in areas with no RS1 expression, as shown by cone arrestin immunostaining. Additional local structural protection and cone cell benefit were observed even in retinas in which overall retinal improvement was not detectable by dark-adapted ERG, suggesting that the gene therapy of the present invention provides additional local functional rescue. Photopic ERG also showed functional rescue correlated to cone cell rescue as measured by arrestin signal. Thus, a novel gene supplementation therapy for the treatment of XLRS, including XLRS caused by missense mutations of RS1, was developed and shown to be effective.

Additional factors contributing to successful gene supplementation therapy were investigated, and it was observed that sufficient WT RS1 expression in photoreceptors, the particular cell type where endogenous RS1 is expressed, including expression level and protein coverage area in the whole retina, are all critical for treatment efficacy. This disclosure demonstrates that critical factors that should be considered in designing a therapeutic approach include the choice of administration route, a specific promoter targeting both photoreceptor cell types, viral serotypes and tropism, window selection of viral vector delivery, and inflammatory response.

IVT delivery is one of the major administration routes for the treatment of posterior ocular diseases because it provides several benefits, such as direct delivery of drugs into the vitreous and retina, and the simplicity of achieving this procedure for medical doctors (Yasukawa et al., 2004, Prog Retin Eye Res, 23:253-281; Gaudana et al., 2010, AAPS J, 12:348-360). However, in gene therapy studies, because of the thickness and structure of the retina, IVT injection has a limited target effect in the posterior segment of the eye. There is no clinical evidence that an IVT-delivered AAV vector can transduce outer retinal cells (photoreceptors/RPE) at sufficient levels to mediate a therapeutic response (Mendell et al., 2021, Mol Ther, 29:464-488). Intravitreally delivered AAV8-RS1 failed to restore retinal structure or function in XLRS patients (Clinical Trials.gov: NCT02317887), partially due to inefficient targeting of RS1 to photoreceptors.

Compared to intravitreal injection, subretinal administration has more direct effects on the targeted cells in the subretinal space, which provides a more precise and efficient route of ocular drug delivery for gene therapies. For example, subretinal administration has been performed effectively for retinitis pigmentosa (RP) and Leber's congenital amaurosis (LCA). In the present gene therapy study in mouse animal models, significant outstanding treatment effects of subretinal delivery, but not IVT delivery, were observed.

However, it is a consensus that subretinal injection requires surgical manipulation of the retina. The fragile retina of XLRS disease makes surgical repair difficult (Ferrone et al., 1997, Am J Ophthalmol, 123:742-747), since the local displacement of the retina from the surgery, even transiently, may aggravate the retinal damage and vision loss. In the present study, OCT imaging of the retinal structure 2 or 4 months after subretinal injection did reveal local scar-like retinal damage, but no functional difference in PBS injected eyes compared to non-injected eyes was observed by ERG. Therefore, the efficacy of the gene therapy by subretinal delivery of the present invention may not be compromised by the procedure. However, differences in retinal structure between rodents and humans must be taken into consideration. The injection location was very peripheral in the mouse eye, while in a human patient the injection should at least cover the macular area, if not exactly at the fovea, to obtain desired treatment effect.

It has been suggested that RS1, expressed either naturally in WT mice or via gene therapy targeting photoreceptors of Rs1 KO mice, is capable of distributing longitudinally and laterally within the retina to its normal extracellular targeting sites (Min et al., 2005, Mol Ther, 16:1010-1017). However, a three-fold higher dose (9×10¹⁰ vg/μL/eye) didn't show significantly enhanced structural or functional improvement (data not shown). This is most likely due to the inability to uniformly deliver the transgene to all photoreceptors and the confounding results that robust RS1 protein expression in a diseased retina (as with the CAG promoter) does not directly translate to a therapeutic benefit. The total transduced photoreceptor cell number and lateral retinal coverage area by these cells are not significantly different between the two tested titers. This implies a limitation of subretinal delivery, which may not provide widespread distribution of the viral particles to transduce all the impaired photoreceptors.

A non-invasive administration of viral vectors for transducing outer retinal cells which provides wider distribution is desirable. Suprachoroidal space (SCS) injection is such a new route of drug administration to deliver therapeutics to the posterior segment, which has been shown to be a safe and efficient procedure offering widespread expression in photoreceptor/RPE for ocular gene therapy with viral and non-viral vectors in both preclinical and clinical research (Chiang et al., 2018; Jung et al., 2019; Kansara et al., 2020, J Ocul Pharmacol Ther, 36:384-392; Wan et al., 2020, Transl Vis Sci Technol, 9:27; Mehta et al., 2021). The present invention may be carried out using SCS injection for gene supplementation therapy to treat XLRS.

It has been previously suggested that transduction of photoreceptors is unnecessary for gene therapy using RS1, because RS1 is a secreted protein that may diffuse between retinal layers. However, the presence of internal barriers such as the inner limiting membrane and compartments within the retina may limit the ability of RS1 to diffuse. The examples disclosed above demonstrate that RS1 expression in photoreceptors, as opposed to other parts of the retinal tissue, is imperative for successful gene therapy. Therefore, it was also demonstrated that the choice of promoter is one of the key contributors to functional RS1 expression and successful therapy.

The comparison of the efficacy of two different promoters, CAG and Rho, showed that structural and functional restoration was achieved in Rho-hRS1- but not CAG-hRS1-injected eyes, despite the fact that RS1 expression 6 weeks after CAG-hRS1 injection was stronger than that from Rho-hRS1, as shown in FIGS. 9A and 9B. The ubiquitous promoter CAG drove RS1 expression in all retinal layers with overall stronger expression, while Rho-driven expression was not as strong but was photoreceptor-specific, with a majority of the expressed RS1 protein localized to the photoreceptor inner segment and functional. These results demonstrate that WT hRS1 used in XLRS treatment must be expressed in photoreceptors, contrary to the previous expectation that hRS1 should function similarly without a specific localization due to being a secreted protein with extracellular functions. It is possible that hRS1 has as-yet-unknown intracellular functions, or that its extracellular functions require precise localization.

It was surprisingly found that gene supplementation of Rho-hRS1 led to improved cone photoreceptor survival, as shown in FIG. 8C. One possible explanation is as follows. First, it has been demonstrated that rod-derived cone viability factor (RdCVF), a truncated thioredoxin-like protein specifically expressed by photoreceptors, is involved in paracrine interaction between rod and cone photoreceptors and has a key role in maintaining cone cell viability (Léveillard et al., 2004, Nature Genetics, 36:755-759). It is possible that rescue of rod cells by gene supplementation therapy may result in a relative increase in diffusible RdCVF secreted by rods, leading to improved conditions for cones even in regions without outstanding RS1 immunosignal in the retinal flat mount of treated RS1 KO mouse, as shown in FIG. 8C. Aït-Ali and colleagues (Aït-Ali et al., 2015, Cell, 161:817-832) further demonstrated that RdCVF promoted retinal cone survival by binding to the cell-surface complex Basigin-1/glucose transporter (BSG1/GLUT1), accelerating the entry of glucose into photoreceptors and enhancing aerobic glycolysis.

Alternatively, in several studies, rhodopsin gene promoters were shown to be “leaky,” allowing transgene expression in both rods and in cones (Glushakova et al., 2006, Mol Vis, 12:298-309; Woodford et al., 1994, Exp Eye Res, 58:631-635; Gouras et al., 1994, Vis Neurosci, 11:1227-1231). Two independent transgenic mouse lines carrying different sizes of bovine rhodopsin promoter fragments fused with lacZ (ß-galactosidase) gene showed reporter gene expression in cones as well as rods, although the level of staining appeared to be less in the cones than in the rods (Gouras et al., 1994). It was claimed that although cones do not express the neural retina leucine zipper protein (NRL) and NR2E3 trans-factors considered necessary for activation of Rhodopsin promoter in rods, other general transcription factors in cones may compensate (Glushakova, et al., 2006). However, in another study, when subretinally delivered by an AAV2/5 vector into mouse eye, human rhodopsin kinase (RK) gene promoter-mediated expression was as efficient as, but appeared more uniform than, mouse rhodopsin promoter (mOps)-mediated expression. In cones, the hRK promoter drove expression, whereas the mOps did not.

An alternative and more robust photoreceptor-specific promoter driving RS1 protein expression in both cone and rod is still desired, which should be meaningful for treating XLRS in human patients by gene therapy since XLRS is characterized by bilateral maculopathy-radial streaks arising from foveal schisis with associated splitting of inner retinal layers in the peripheral retina in 50% of patients. All patients studied in Molday et al., 2012, were affected by foveal schisis, which had the main negative impact on quality of life. Adaptive optics scanning laser ophthalmoscopy (AOSLO) images revealed increased cone spacing and abnormal packing in the macula of patients with molecularly characterized XLRS (Duncan et al., 2011, Invest Ophthalmol Vis Sci, 52:9614-9623). To achieve successful RS1 gene therapy, specific promoters targeting both rods and cones are one of the essential aspects to be explored.

7m8, AAV5, and AAV8 serotypes were compared in the present study (data not shown). When used for subretinal delivery of Rho-RS1, similar functional and structural recovery was observed with each of the tested capsids. A person of skill in the art may select an AAV serotype according to their particular needs.

Although it is clear from the results presented above that virally delivered WT RS1 protein can provide some therapeutic benefit in disease variant and KO mice, additional study is warranted regarding the quantitative and qualitative requirements concerning the extent of expression necessary for substantial benefit, which may be attributed to the optimal tropism of the capsid, robust and specific promoter, all or near all photoreceptors transduced, and the optimal titer of the viral vector. An independent approach by generating controlled over-expression of RS1 transgenic mice in the background of mutant Rs1 with disease phenotype can be utilized to offer correlation between the extent of phenotype improvement and transgene expression level.

Timing of treatment is an additional factor to optimize. The multicenter retrospective study with linear mixed models (Boon et al., 2021) revealed a slow annual decline of 0.39% in BCVA with a relatively stable visual acuity until the age of 20 years, suggesting an optimal window of opportunity for treatment within the first two decades of life. In the presently described preclinical testing in mouse models, subretinal injection was evaluated at P14, P21, and 5.5 weeks of age. Little difference in efficacy was observed, suggesting that the chosen window for the majority of the presently described gene therapy studies, focusing on ˜P21, was effective. Although delivery of therapeutic gene to Rs1 animal models older than 5.5 weeks of age was not attempted, it is presumed that the treatment efficacy would reduced if treatment were delayed to 3-4 months old, since the retinal degeneration is severe at this age (data not shown), corresponding to human patients at their twenties.

The two clinical programs on XLRS gene therapy sponsored by NEI (AAV8-hRS1) and AGTC (AAV2-hRS1) were both unsuccessful. The clinical programs used vectors less penetrant than 7m8, which, as demonstrated in the examples above, is not even penetrant enough to function in mice when administered by intravitreal injection. Therefore, it is possible that a reason the clinical programs failed was due to a suboptimal choice of vector, promoter, administration route, and/or timing of administration.

In summary, the present disclosure demonstrates that several key factors are important to improve the treatment efficacy of gene supplementation therapy in animal models of XLRS: RS1 expression level, cell types expressing the transgene, and therapeutic protein spatial distribution and coverage. Therefore, routes of delivery, viral serotypes and tropism, promoter specificity and robustness, and window selection of viral vector delivery are all to be considered in further exploration of preclinical gene therapy for XLRS.

What is claimed is:

1. A method for treating retinal degeneration caused by one or more missense mutations of RS1, comprising administering a vector including a gene encoding a functional RS1 protein to a subject.

2. The method of claim 1, wherein said vector comprises an AAV.

3. The method of claim 2, wherein said AAV is selected from a group consisting of AAV2, AAV5, AAV8, AAV9, a modified version of AAV2, a modified version of AAV5, a modified version of AAV8, a modified version of AAV9, and a combination thereof.

4. The method of claim 3, wherein said AAV is AAV2.

5. The method of claim 1, wherein said vector further includes a promoter, wherein said promoter drives the expression of said gene in the retina.

6. The method of claim 5, wherein said promoter is selected from a group consisting of a rhodopsin kinase promoter, a PR2.1 promoter, a PR1.7 promoter, or an IRBP promoter.

7. The method of claim 6, wherein said promoter comprises a rhodopsin kinase promoter.

8. The method of claim 1, wherein said administration comprises subretinal injection.

9. The method of claim 1, wherein said administration comprises suprachoroidal space injection.

10. The method of claim 1, wherein said one or more missense mutations are selected from a group consisting of L13P, C38S, C40S, C42S, C59S, C63S, E72K, S73P, C83S, W96R, R102W, R102Q, G109E, G109R, C110S, C110Y, L127P, I136T, R141H, C142S, C142R, C142W, D143V, N163Y, N179D, P192S, P192T, P193S, P203L, R213W, C219S, C219R, C219W, C219G, C223S, C223R, and C223Y.

11. The method of claim 1, wherein said one or more missense mutations comprise C59S.

12. The method of claim 1, wherein said one or more missense mutations comprise R141C.

13. The method of claim 1, further comprising restoration or partial restoration of retinal structure, wherein restoration or partial restoration of retinal structure is measured using optical coherence tomography.

14. The method of claim 1, further comprising restoration or partial restoration of retinal function, wherein restoration or partial restoration of retinal function is measured using electroretinography.

15. A method for treating retinal degeneration caused by one or more missense mutations of RS1, comprising administering an AAV2 vector including a rhodopsin kinase promoter and a gene encoding a functional RS1 protein.

16. The method of claim 15, wherein said administration comprises subretinal injection.

17. The method of claim 15, wherein said one or more missense mutations comprise C59S.

18. The method of claim 15, wherein said one or more missense mutations comprise R141C.

19. The method of claim 15, further comprising assessing the restoration of retinal structure using optical coherence tomography.

20. The method of claim 15, further comprising assessing the restoration of retinal function using electroretinography.
